(19) United States
(12) Patent Application Publication
(10) Pub. No.: US 2003/0082648 A1
(43) Pub. Date: May 1, 2003

(54) Novel mass receptor-analogous protein and dnas thereof

(76) Inventors: Shuji Hinuma, Tsukuba-shi (JP); Shoji Fukusumi, Ibaraki (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/221,841

(22) PCT Filed: Mar. 15, 2001
(86) PCT No.: PCT/JP01/02053

(30) Foreign Application Priority Data
Mar. 17, 2000 (JP) 2000-81835
Dec. 11, 2000 (JP) 2000-381698

(51) Int. Cl. G01N 33/567; A61K 38/17; C07H 21/04; C12P 21/02; C12N 5/06

(52) U.S. Cl. 435/7.21; 435/69.1; 435/320.1; 435/325; 530/350; 536/23.5; 514/12; 530/388.22

(57) ABSTRACT

The present invention provides novel G protein-coupled receptor proteins derived from mouse heart and rat whole brain, which are useful in screening agonists/antagonists, etc., DNAs encoding these proteins, and the like.

DNAs encoding the G protein-coupled receptor proteins derived from mouse heart and rat whole brain, or salts thereof, are useful for constructing a recombinant receptor protein expression system, developing a receptor-binding system with the use of this expression system, screening candidate compounds for drugs, etc.

### FIELD OF THE INVENTION

The present invention relates to novel mas receptor-analogous proteins derived from mouse heart and rat whole brain, or salts thereof, DNAs encoding the same, and the like.

### BACKGROUND ART

Many physiologically active substances like hormones, neurotransmitters, etc. regulate the functions of the body via specific receptor proteins present on cell membranes. Most of these receptor proteins are coupled to guanine nucleotide-binding proteins (hereinafter sometimes referred to as G proteins) to mediate the intracellular signal transduction through activation of the G proteins. These receptor proteins possess a common structure comprising seven transmembrane domains and are thus referred to collectively as G protein-coupled receptor proteins or seven transmembrane receptor proteins (7 TMR).

G protein-coupled receptor proteins exist on cells of a living body and each functional cell surface of organs and play very important roles as the targets of molecules, for example, hormones, neurotransmitters, physiologically active substances and the like, which molecules regulate the functions of cells and organs in vivo. These receptors mediate signal transduction in a cell by binding to physiologically active substances and various reactions such as activation or inhibition of cells, are induced.

To clarify the relationship between substances that regulate complicated biological functions in various cells and organs and their specific receptor proteins, in particular, G protein-coupled receptor proteins would elucidate the functional mechanisms in various cells and organs in the body to provide a very important means for developing drugs closely associated with these functions.

For example, in various organs, their physiological functions are controlled in vivo through regulation by many hormones, hormone-like substances, neurotransmitters or physiologically active substances. In particular, physiologically active substances are found in numerous sites of the body and regulate the physiological functions through their corresponding receptor proteins. However, many unknown hormones, neurotransmitters or other physiologically active substances still exist in the body and, as to their receptor proteins, most of their structures have not yet been reported. Moreover, it is still unknown if there are subtypes of known receptor proteins.

In these seven transmembrane receptor proteins, a mas receptor that is isolated and identified as one of oncogenes is known. The mas receptor is an orphan receptor, the ligand of which is unidentified yet. The mas receptor is reported in association with cancer (Cell, 1986, June 6; 45 (5): 711-9, Isolation and characterization of a new cellular oncogene encoding a protein with multiple potential transmembrane domains. Young, D., Waitches, G., Birchmeier, C., Fasano, O., Wigler, M.), on similarity to an angiotensin receptor (Nature, 1988, September 29; 335 (6189): 437-40, The mas oncogene encodes an angiotensin receptor. Jackson, T. R., Blair, L. A., Marshall, J., Goedert, M., Hanley, M. R.) and, in association with memory (J. Biol. Chem., 1998, May 8; 273 (19): 11867-73, Sustained long term potentiation and anxiety in mice lacking the Mas protooncogene. Walther, t., Balschun, D., Voigt, J. P., Fink, H., Zuschratter, W., Birchmeier, C., Ganten, D., Bader, M.), suggesting that among receptors, the mas receptor would be involved in especially important physiological roles. However, any receptor showing homology as high as 40% or more to this mas receptor is unknown.

Clarification of the relationship between substances that regulate complicated biological functions and their specific receptor proteins is a very important means for development of pharmaceuticals. Furthermore, for efficient screening of agonists and antagonists to receptor proteins in developing pharmaceuticals, it was necessary to elucidate the functions of biologically expressed receptor protein genes and to express the genes in appropriate expression systems.

In recent years, random analysis of cDNA sequences has been extensively studied as a means for analysis of expressed genes in vivo. The sequences of cDNA fragments thus obtained have been registered and disclosed to the public on databases as Expressed Sequence Tag (EST). However, since many ESTs contain sequence information only, it is difficult to predict their functions.

Heretofore, substances that inhibit the binding of G protein-coupled receptors to physiologically active substances (i.e., ligands) or substances that bind and induce signal transduction similar to that induced by physiological active substances (i.e., ligands) have been used as pharmaceuticals in terms of antagonists and agonists specific to the receptors that regulate the biological functions. Thus, discovery of a novel G protein-coupled receptor protein that can be targeted for pharmaceutical development and cloning of its gene (e.g., cDNA) are very important means in search for a specific ligand, agonist, and antagonist of the novel G protein-coupled receptor protein.

However, not all G protein-coupled receptors have been discovered. Even now, there are unknown G protein-coupled receptors and many receptors or so-called orphan receptors, in which the corresponding ligands are yet unidentified. Therefore, search of a novel G protein-coupled receptor and elucidation of its functions are awaited.

G protein-coupled receptors are useful in search for a novel physiological active substance (i.e., a ligand) using the signal transduction activity as the indicator and in search for agonists and antagonists to the receptor. On the other hand, even if no physiological ligand is found, agonists and antagonist to the receptor may be prepared by analyzing the physiological activity of the receptor through inactivation experiment of the receptor (knockout animal). Ligands, agonists, antagonists, etc. to the receptor are expected to be used as prophylactic/therapeutic and diagnostic agents for diseases associated with dysfunction of the G protein-coupled receptor.

Very often hypofunction or hyperfunction of the G protein-coupled receptor due to genetic variation of the receptor in vivo becomes causes for some disorders. In this case, the G protein-coupled receptor may be used not only for administration of antagonists or agonists to the receptor, but also for gene therapy by transfer of the receptor gene into the body (or certain specific organs) or by transfer of the antisense nucleic acid to the receptor gene. In such a gene therapy, information on the base sequence of the receptor is essentially required to examine the deletion or mutation on the gene. The receptor gene is also applicable as prophylactic/therapeutic drugs and diagnostic agents for diseases associated with dysfunction of the receptor.

The present invention provides a novel and useful G protein-coupled receptor protein as described above. That is, the present invention provides a novel G protein-coupled receptor protein, its partial peptides or salts thereof, as well as polynucleotides (DNAs, RNAs and derivatives thereof) containing polynucleotides (DNAs, RNAs and derivatives thereof) encoding the G protein-coupled receptor protein or its partial peptides, recombinant vectors containing the polynucleotides, transformants bearing the recombinant vectors, methods of manufacturing the G protein-coupled receptor protein or salts thereof, antibodies to the G protein-coupled receptor protein, its partial peptides or salts thereof, compounds that alter the expression level of said G protein-coupled receptor protein, methods of determination of ligands to the G protein-coupled receptor protein, methods of screening compounds (antagonists or agonists) or salts thereof that alter the binding property between ligands and the G protein-coupled receptor protein, kits for use in the screening methods, compounds (antagonists or agonists) or salts thereof that alter the binding property between ligands and the G protein-coupled receptor protein obtainable by the screening methods or using the screening kit, and pharmaceutical compositions comprising the compounds (antagonists or agonists) that alter the binding property of ligands to the G protein-coupled receptor protein, or compounds or salts thereof that alter the expression level of the G protein-coupled receptor protein, and the like.

### DISCLOSURE OF THE INVENTION

The inventors performed extensive studies and as a result, succeeded in isolation of cDNAs encoding the novel protein derived from the mouse heart and from the whole rat brain and in analysis of the full-length base sequence of the cDNAs. The amino acid sequence deduced from the base sequence has supported that the first to seven transmembrane regions were observed on the hydrophobic plotting analysis, confirming that the proteins encoded by these cDNA are seven transmembrane receptor proteins. Based on these findings, the inventors have made further investigations and as a result, have accomplished the present invention.

Therefore, the present invention relates to:

(1) A protein containing the same or substantially the same amino acid sequence as the amino acid sequence represented by SEQ ID NO: 1, or salts thereof;

(2) The protein or salts thereof according to (1), wherein substantially the same amino acid sequence is represented by SEQ ID NO: 5;

(3) A partial peptide of the protein according to (1), its esters or amides, or salts thereof;

(4) A polynucleotide containing a polynucleotide encoding the protein according to (1) or the partial peptide according to (3);

(5) The polynucleotide according to (4), which is a DNA;

(6) The polynucleotide according to (4), which has the base sequence represented by SEQ ID NO: 2 or SEQ ID NO: 6;

(7) A recombinant vector containing the polynucleotide according to (4);

(8) A transformant transformed by the recombinant vector according to (7);

(9) A method of manufacturing the protein or salts thereof according to (1) or the partial peptide, its amides or esters, or salts thereof according to (3), which comprises culturing the transformant according to (8) and producing the protein according to (1) or the partial peptide according to (3);

(10) An antibody to the protein or salts thereof according to (1) or to the partial peptide, its esters or amides, or salts thereof according to (3);

(11) The antibody according to (10), which is a neutralizing antibody to inactivate signal transduction of the protein according to (1);

(12) A diagnostic product comprising the antibody according to (10);

(13) A ligand to the protein or salts thereof according to (1) or to the partial peptide, its esters or amides, or salts thereof according to (3), which is obtainable using the protein or salts thereof according to (1) or using the partial peptide, its esters or amides, or salts thereof according to (3);

(14) A pharmaceutical composition comprising the ligand according to (13);

(15) A method of determining the ligand to the protein or salts thereof according to (1) or to the partial peptide, its esters or amides, or salts thereof according to (3), which comprises using the protein or salts thereof according to (1) or the partial peptide, its esters or amides, or salts thereof according to (3);

(16) A method of screening a compound or salts thereof that alter the binding property between a ligand and the protein or salts thereof according to (1) or the partial peptide, its esters or amides, or salts thereof according to (3), which comprises using the protein or salts thereof according to (1), or the partial peptide, its esters or amides, or salts thereof according to (3);

(17) A kit for screening a compound or salts thereof that alter the binding property between a ligand and the protein or salts thereof according to (1) or the partial peptide, its esters or amides, or salts thereof according to (3), comprising the protein or salts thereof according to (1), or the partial peptide, its esters or amides, or salts thereof according to (3);

(18) A compound or salts thereof that alter the binding property between a ligand and the protein or salts thereof according to (1) or the partial peptide, its esters or amides, or salts thereof according to (3), which is obtainable using the screening method according to (16) or the screening kit according to (17);

(19) A pharmaceutical composition comprising the compound or salts thereof according to (18);

(20) A polynucleotide hybridizable to the polynucleotide according to (4) under high stringent conditions.

(21) A polynucleotide comprising a base sequence complimentary to the polynucleotide according to (4), or a part of the base sequence.

(22) A method of quantifying mRNA of the protein according to (1), which comprises using the polynucleotide according to (4), or a part of the polynucleotide;

(23) A method of quantifying the protein according to (1) or the partial peptide, its amides or esters according to (3), or salts thereof, which comprises using the antibody according to (10);

(24) A method for diagnosis of diseases associated with the functions of the protein according to (1), which comprises using the quantifying method according to (22) or (23);

(25) A method of screening a compound or salts thereof that alter the expression level of the protein according to (1), which comprises using the quantifying method according to (22) or (23); and,

(26) A method of screening a compound or salts thereof that alter the amount of the protein according to (1) on a cell membrane, which comprises using the quantifying method according to (23); and the like.

The present invention further provides:

(27) A protein or salts thereof according to (1), wherein the protein is a protein containing: (i) an amino acid sequence represented by SEQ ID NO:1 or SEQ ID NO:5, (ii) an amino acid sequence represented by SEQ ID NO: 1 or SEQ ID NO:5, of which 1, 2 or more amino acids (preferably about 1 to about 30 amino acids, more preferably about 1 to about 10 amino acids, and most preferably several (1 to 5) amino acids) are deleted; (iii) an amino acid sequence represented by SEQ ID NO: 1 or SEQ ID NO:5, to which 1, 2 or more amino acids (preferably about 1 to about 30 amino acids, more preferably about 1 to about 10 amino acids, and most preferably several (1 to 5) amino acids) are added; (iv) an amino acid sequence represented by SEQ ID NO: 1 or SEQ ID NO:5, in which 1, 2 or more amino acids (preferably about 1 to about 30 amino acids, more preferably about 1 to about 10 amino acids, and most preferably several (1 to 5) amino acids) are substituted by other amino acids; and (v) a combination of the above amino acid sequences;

(28) A method of determining the ligand according to (15), which comprises contacting the protein or salts thereof according to (1) or the partial peptide, its amides or esters, or salts thereof according to (3) with a test compound;

(29) The method of determining the ligand according to (28), wherein the ligand is, for example, angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal polypeptide), somatostatin, dopamine, motilin, amylin, bradykinin, CGRP (calcitonin gene-related peptide), leukotrienes, pancreastatin, prostaglandins, thromboxane, adenosine, adrenaline, α and β-chemokines (e.g., IL-8, GROα, GROβ, GROγ, NAP-2, ENA-78, PF4, IP10, GCP-2, MCP-1, HC14, MCP-3, I-309, MIP-1α, MIP-1 β, RANTES, etc.), endothelin, enterogastrin, histamine, neurotensin, TRH, pancreatic polypeptide, galanin or rat cortistatin;

(30) The method of screening according to (16), wherein comparison is made between (i) the case when the protein or salts thereof according to (1) or the partial peptide, its amides or esters, or salts thereof according to (3) are brought in contact with the ligand and (ii) the case when the protein or salts thereof according to (1) or the partial peptide, its amides or esters, or salts thereof according to (3) are brought in contact with the ligand and a test compound;

(31) A method of screening a compound or salts thereof that alter the binding property between a ligand and the protein or salts thereof according to (1) or the partial peptide, its amides or esters, or salts thereof according to (3), which comprises measuring amounts of labeled ligand bound to the protein or salts thereof according to (1) or the partial peptide, its amides or esters, or salts thereof according to (3), (i) when the labeled ligand is brought in contact with the protein or salts thereof according to (1) or the partial peptide, its amides or esters, or salts thereof according to (3) and (ii) when the labeled ligand and a test compound are brought in contact with the protein or salts thereof according to (1) or the partial peptide, its amides or esters, or salts thereof according to (3) and comparing the amounts measured in (i) and (ii);

(32) A method of screening a compound or salts thereof that alter the binding property between a ligand and the protein or salts thereof according to (1), which comprises measuring amounts of a labeled ligand bound to a cell containing the protein according to (1), (i) when the labeled ligand is brought in contact with the cell, and (ii) when the labeled ligand and a test compound are brought in contact with the cell, and comparing the amounts measured in (i) and (ii);

(33) A method of screening a compound or salts thereof that alter the binding property between a ligand and the protein or salts thereof according to (1), which comprises measuring amounts of a labeled ligand bound to a membrane fraction of the cell containing the protein according to (1), (i) when the labeled ligand is brought in contact with the cell membrane fraction, and (ii) when the labeled ligand and a test compound are brought in contact with the cell membrane fraction, and comparing the amounts measured in (i) and (ii);

(34) A method of screening a compound or salts thereof that alter the binding property between a ligand and the protein or salts thereof according to (1), which comprises measuring amounts of a labeled ligand bound to a protein expressed on a cell membrane of the transformant according to (8) by culturing the transformant, (i) when the labeled ligand is brought in contact with the protein expressed and (ii) when the labeled ligand and a test compound are brought in contact with the protein expressed on a cell membrane of the transformant according to (8) by culturing the transformant, and comparing the amounts measured in (i) and (ii);

(35) A method of screening a compound or salts thereof that alter the binding property between a ligand and the protein or salts thereof according to (1), which comprises measuring protein-mediated cell stimulating activities (i) when a compound that activates the protein or salts thereof according to (1) is brought in contact with a cell containing the protein according to (1) and (ii) when a compound that activates the protein or salts thereof according to (1) and a test compound are brought in contact with a cell containing the protein according to (1), and comparing the activities measured in (i) and (ii);

(36) A method of screening a compound or salts thereof that alter the binding property between a ligand and the protein or salts thereof according to (1), which comprises protein-mediated cell stimulating activities, (i) when a compound that activates the protein or salts thereof according to (1) is brought in contact with a protein expressed on a cell membrane of the transformant according to (8) by culturing the transformant and (ii) when a compound that activates the protein or salts thereof according to (1) and a test compound are brought in contact with a protein expressed on a cell membrane of the transformant according to (8) by culturing the transformant, and comparing the activities measured in (i) and (ii);

(37) The screening method according to (35) or (36), wherein the compound that activates the protein according to (1) is angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal polypeptide), somatostatin, dopamine, motilin, amylin, bradykinin, CGRP (calcitonin gene-related peptide), leukotrienes, pancreastatin, prostaglandins, thromboxane, adenosine, adrenaline, α and β-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic polypeptide, galanin or rat cortistatin;

(38) A compound or salts thereof that alter the binding property between a ligand and the protein or salts thereof according to (1), which is obtainable by the screening method according to (30) through (37);

(39) A pharmaceutical composition comprising a compound or salts thereof that alter the binding property between a ligand and the protein or salts thereof according to (1), which is obtainable by the screening method according to (30) through (37);

(40) A kit for screening according to (17), comprising a cell containing the protein according to (1);

(41) A kit for screening according to (17), comprising a cell membrane fraction of cell containing the protein according to (1);

(42) A kit for screening according to (17), comprising a protein expressed on a cell membrane of the transformant according to (8) by culturing the transformant;

(43) A compound or salts thereof that alter the binding property between a ligand and the protein or salts thereof according to (1), which is obtainable using the screening kit according to (40) through (42);

(44) A pharmaceutical composition comprising a compound or salts thereof that alter the binding property between a ligand and the protein or salts thereof according to (1), which is obtainable using the screening kit according to (40) through (42);

(45) A method of quantifying the protein or salts thereof according to (1), or the partial peptide, its amides or esters, or salts thereof according to (3), which comprises contacting the antibody according to (10) with the protein or salts thereof according to (1) or partial peptide, amides thereof, esters thereof or salts according to (3);

(46) A method of quantifying the protein or salts thereof according to (1), or the partial peptide, its amides or esters, or salts thereof according to (3), in a specimen solution, which comprises competitively reacting the antibody according to (10) with a specimen solution and the labeled protein or salts thereof according to (1) or the labeled the partial peptide, its amides or esters, or salts thereof according to (3); and measuring the ratio of the labeled form of the protein or salts thereof according to (1) or the partial peptide, its amides or esters, or salts thereof according to (3), which are bound to the antibody; and,

(47) A method of quantifying the protein or salts thereof according to (1) or the partial peptide, its amides or esters, or salts thereof according to (3), in a specimen solution, which comprises reacting a specimen solution simultaneously or sequentially with the antibody according to (10) immobilized on a carrier and a labeled form of the antibody according to (10), and then measuring the activity of a labeling agent on the immobilized carrier.

### BRIEF DESCRIPTION OF THE DRAWINGS

FIG. 1 shows comparison in amino acid sequences between mouse type mas receptor and the receptor protein (mouse type ML: SEQ ID NO:1) of the present invention, wherein mmas.PRO and mML1.PRO display the amino acid sequences of mouse type mas receptor and the amino acid sequence of mouse type novel receptor acquired this time, respectively, and the same amino acids as the sequence of the mas receptor are surrounded by a box.

FIG. 2 shows the hydrophobic plotting of the receptor protein of the present invention.

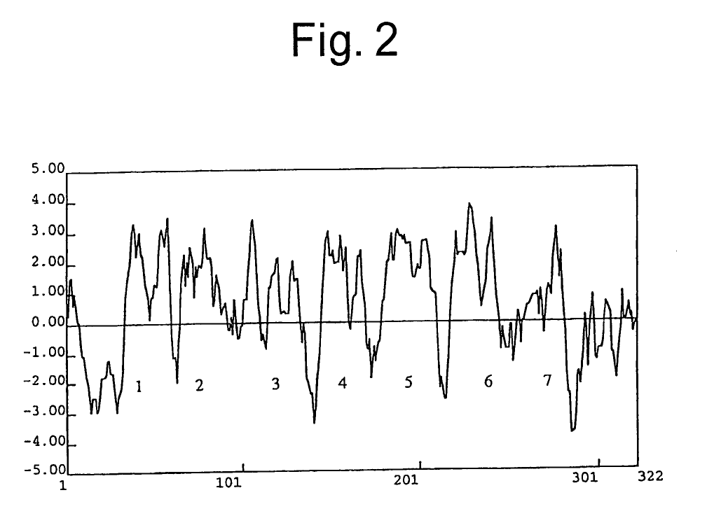

FIG. 3 shows comparison in amino acid sequences among mouse type ML receptor (SEQ ID NO:1), rat type ML receptor (SEQ ID NO:5), rat type mas receptor and mouse type mas receptor, wherein mML.pro, rML.pro, mMAS.PRO and rMAS.Pro display the amino acid sequences of mouse type ML receptor, rat type ML receptor acquired this time, mouse type mas receptor and rat type mas receptor, respectively, and the same amino acids are surrounded by a box.

FIG. 4 indicates the assay results of the activity of releasing arachidonic acid metabolites from CHO-mML cells and mock CHO cells. By addition of 1×10⁻⁵ M rat cortistatin, the activity of increasing the amount of specific arachidonic acid metabolites released from CHO-mML cells was noted.

In the figure, ▪ and □ display the arachidonic acid metabolite-releasing activity from CHO-mML cells and the arachidonic acid metabolite-releasing activity from mock CHO cells, respectively.

### BEST MODE FOR CARRYING OUT THE INVENTION

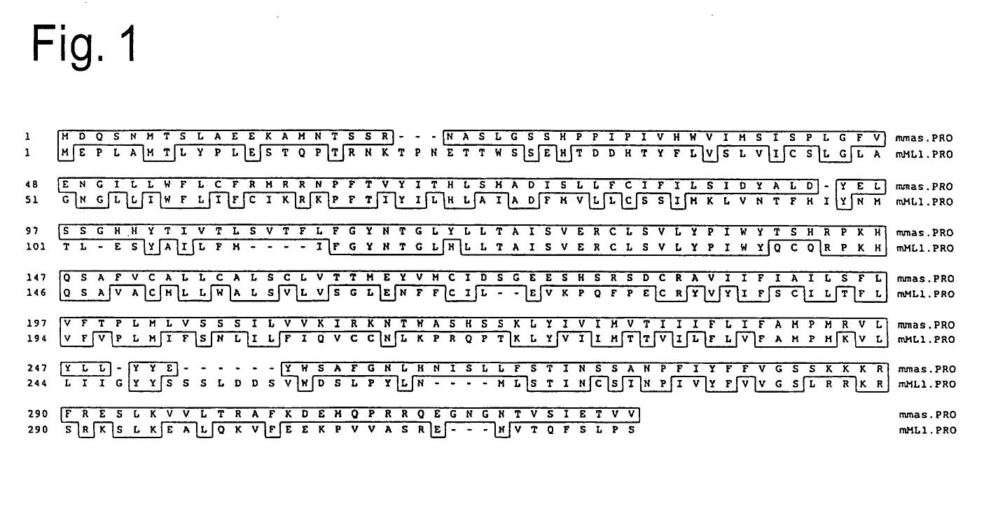

The G protein-coupled receptor protein (hereinafter sometimes simply referred to as the receptor protein) of the present invention is a receptor protein containing the same or substantially the same amino acid sequence presented by the amino acid sequence represented by SEQ ID NO: 1 (the amino acid sequence shown in FIG. 1).

The receptor protein of the present invention may be derived from any type of cells of mammals (e.g., human, guinea pigs, rats, mice, rabbits, swine, sheep, bovine, monkeys, etc.) (e.g., splenocytes, nerve cells, glial cells, β cells of pancreas, bone marrow cells, mesangial cells, Langerhans' cells, epidermic cells, epithelial cells, endothelial cells, fibroblasts, fibrocytes, myocytes, fat cells, immune cells (e.g., macrophage, T cells, B cells, natural killer cells, mast cells, neutrophils, basophils, eosinophils, monocytes), megakaryocytes, synovial cells, chondrocytes, bone cells, osteoblasts, osteoclasts, mammary gland cells, hepatocytes or interstitial cells; or the corresponding precursor cells, stem cells, cancer cells, etc.) or hemocyte type cells; or any tissues where such cells are present, such as brain or any of brain regions (e.g., olfactory bulb, amygdaloid nucleus, basal ganglia, hippocampus, thalamus, hypothalamus, subthalamic nucleus, cerebral cortex, medulla oblongata, cerebellum, occipital pole, frontal lobe, temporal lobe, putamen, caudate nucleus, corpus callosum, substantia nigra), spinal cord, hypophysis, stomach, pancreas, kidney, liver, gonad, thyroid, gall-bladder, bone marrow, adrenal gland, skin, muscle, lung, gastrointestinal tract (e.g., large intestine and small intestine), blood vessel, heart, thymus, spleen, submandibular gland, peripheral blood, peripheral blood cells, prostate, testis, ovary, placenta, uterus, bone, joint, skeletal muscle, etc. (especially brain or any of brain regions). The receptor protein may also be a synthetic protein.

The amino acid sequence which has substantially the same amino acid sequence as the amino acid sequence represented by SEQ ID NO: 1 includes an amino acid sequence having at least about 50% homology, preferably at least about 70% homology, more preferably at least about 80% homology, much more preferably at least about 90% homology and most preferably at least about 95% homology, to the amino acid sequence represented by SEQ ID NO:1.

Examples of the protein which has substantially the same amino acid sequence as the amino acid sequence shown by SEQ ID NO:1 include a protein containing substantially the same amino acid sequence as the amino acid sequence shown by SEQ ID NO:1 and having an activity substantially equivalent to that of the amino acid sequence represented by SEQ ID NO:1, and the like.

The substantially equivalent activities are, for example, a ligand binding activity, a signal transduction activity, etc. The term substantially equivalent is used to mean that the nature of these activities is equivalent. Therefore, it is preferred that these activities such as a ligand binding activity, a signal transduction activity, etc. are equivalent in strength (e.g., about 0.01 to about 100 times, preferably about 0.5 to about 20 times, more preferably about 0.5 to about 2 times), and it is allowable that even differences among grades such as the strength of these activities, molecular weight of the polypeptide, etc., may be present.

The activities such as a ligand binding activity, a signal transduction activity, or the like can be assayed according to a publicly known method, for example, by means of ligand determination or screening, which will be later described.

Specific examples of substantially the same amino acid sequence as the amino acid sequence represented by SEQ ID NO:1 are the amino acid sequence shown by SEQ ID NO:5, and the like.

Specific examples of the receptor protein of the present invention include proteins containing (i) an amino acid sequence represented by SEQ ID NO: 1 or SEQ ID NO:5, of which 1, 2 or more amino acids (preferably about 1 to about 30 amino acids, more preferably about 1 to about 10 amino acids, and most preferably several (1 to 5) amino acids) are deleted; (ii) an amino acid sequence represented by SEQ ID NO: 1 or SEQ ID NO:5, to which 1, 2 or more amino acids (preferably about 1 to about 30 amino acids, more preferably about 1 to about 10 amino acids, and most preferably several (1 to 5) amino acids) are added; (iii) an amino acid sequence represented by SEQ ID NO: 1 or SEQ ID NO:5, in which 1, 2 or more amino acids (preferably about 1 to about 30 amino acids, more preferably about 1 to about 10 amino acids, and most preferably several (1 to 5) amino acids) are substituted by other amino acids; or (iv) a combination of the above amino acid sequences, and the like.

Throughout the present specification, the receptor proteins are represented in accordance with the conventional way of describing peptides, that is, the N-terminus (amino terminus) at the left hand and the C-terminus (carboxyl terminus) at the right hand. In the receptor proteins of the present invention including the receptor proteins containing the amino acid sequence shown by SEQ ID NO:1, the C-terminus is usually in the form of a carboxyl group (—COOH) or a carboxylate (—COO⁻) but may be in the form of an amide (—CONH₂) or an ester (—COOR).

Examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; an aralkyl having 7 to 14 carbon atoms such as a phenyl-C₁₋₂ alkyl group, e.g., benzyl, phenethyl, etc.; an α-naphthyl-C₁₋₂ alkyl group such as α-naphthylmethyl, etc.; and the like. In addition, pivaloyloxymethyl or the like, which is used widely as an ester for oral administration may also be used.

Where the receptor protein of the present invention contains a carboxyl group (or a carboxylate) at a position other than the C-terminus, it may be amidated or esterified and such an amide or ester is also included within the receptor protein of the present invention. The ester group may be the same group as that described with respect to the above C-terminal.

Furthermore, examples of the receptor protein of the present invention include variants of the above receptor protein, wherein the amino group at the N-terminus (e.g., methionine residue) of the peptide is protected with a protecting group (e.g., a C₁₋₆ acyl group, e.g., formyl group, a C₂₋₆ alkanoyl group such as acetyl group, etc.); those wherein the N-terminal region is cleaved in vivo and the glutamyl group thus formed is pyroglutaminated; those wherein a substituent (e.g., —OH, —SH, amino group, imidazole group, indole group, guanidino group, etc.) on the side chain of an amino acid in the molecule is protected with a suitable protecting group (e.g., a C₁₋₆ acyl group, e.g., formyl group, a C₂₋₆ alkanoyl group such as acetyl group, etc.), or conjugated proteins such as glycoproteins having sugar chains.

Specific examples of the receptor protein of the present invention include a mouse-derived (preferably mouse heart-derived) receptor protein containing the amino acid sequence represented by SEQ ID NO:1, a rat-derived (preferably rat whole brain-derived) receptor protein containing the amino acid sequence represented by SEQ ID NO:5, etc.

As the partial peptide of the receptor protein of the present invention (hereinafter sometimes merely referred to as the partial peptide of the present invention), any partial peptide of the receptor protein described above may be used. In the receptor protein molecule of the present invention, for example, a part of which is exposed to the outside of a cell membrane and has a receptor binding activity, or the like may be employed.

Specifically, the partial peptide of the receptor protein having the amino acid sequence represented by SEQ ID NO:1 is a peptide containing the part which is found to be an extracellular domain (a hydrophilic domain) in the hydrophobic plotting analysis. A peptide containing a hydrophobic domain part can be used as well. In addition, the peptide may contain each domain separately or plural domains together.

As the partial peptide of the present invention, preferred is a peptide having the sequence of at least 20, preferably at least 50 and more preferably at least 100 amino acids, in the amino acid sequence, which constitutes the receptor protein of the present invention.

The substantially the same amino acid sequence includes an amino acid sequence having at least about 50% homology, preferably at least about 70% homology, more preferably at least about 80% homology, much more preferably at least about 90% homology and most preferably at least about 95% homology, to these amino acid sequences.

Herein the term “substantially equivalent activity” refers to the same significance as defined hereinabove. The “substantially equivalent activity” can be assayed by the same method as described above.

In the partial peptide of the present invention, at least 1 or 2 (preferably about 1 to about 10, more preferably several (1 to 5)) amino acids may be deleted in the amino acid sequence described; at least 1 or 2 (preferably about 1 to about 20, more preferably about 1 to about 10, much more preferably several (1 to 5)) amino acids may be added to the amino acid sequence; or at least 1 or 2 (preferably about 1 to about 10, more preferably several, much more preferably about 1 to about 5) amino acids may be substituted by other amino acids in the amino acid sequence.

In the partial peptide of the present invention, the C-terminus is usually in the form of a carboxyl group (—COOH) or a carboxylate (—COO⁻) but may be in the form of an amide (—CONH₂) or an ester (—COOR), as in the polypeptide of the present invention described above. Herein, R for the ester has the same significance as described above.

Where the partial peptide of the present invention contains a carboxyl group (or a carboxylate) at a position other than the C-terminus, it may be amidated or esterified and such an amide or ester is also included within the partial peptide of the present invention. The ester group used may be the same group as that described with respect to the above C-terminal ester, etc.

The partial peptide of the present invention further includes those in which the amino group of the N-terminal methionine residue is protected by a protecting group, those in which the N-terminal end is cleaved in vivo and the produced Gln is pyroglutamated, those in which substituents on the side chains of amino acids are intramolecularly protected by appropriate protecting groups, or those in which sugar chains are bound, namely, so-called glycopeptides, and the like.

As the salts of the receptor protein of the present invention, or its partial peptides, there are salts with physiologically acceptable acids or bases, with particular preference in the form of physiologically acceptable acid addition salts. Examples of such salts are salts with inorganic acids (e.g., hydrochloric acid, phosphoric acid, hydrobromic acid, sulfuric acid), salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

The receptor protein of the present invention or salts thereof may be manufactured by a publicly known method used to purify a receptor protein from human or other mammalian cells or tissues described above, or may also be manufactured by culturing a transformant containing a DNA encoding the receptor protein of the present invention, as will be later described. Furthermore, the receptor protein or salts thereof may also be manufactured by protein synthesis, which will be described hereinafter, or by its modifications.

Where the receptor protein or salts thereof are manufactured from human or mammalian tissues or cells, human or mammalian tissues or cells are homogenized and extracted with an acid or the like, and the extract is purified and isolated by a combination of chromatography techniques such as reversed phase chromatography, ion exchange chromatography, and the like.

To synthesize the receptor protein of the present invention, its partial peptide or its salts or amides, commercially available resins that are used for protein synthesis may be used. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmethylphenyl acetamidomethyl resin, polyacrylamide resin, 4-(2′,4′-dimethoxyphenyl-hydroxymethyl)phenoxy resin, 4-(2′,4′-dimethoxyphenyl-Fmoc-aminoethyl) phenoxy resin, etc. Using these resins, amino acids, in which α-amino groups and functional groups on the side chains are appropriately protected, are condensed on the resin in the order of the sequences of the objective protein according to various condensation methods publicly known in the art. At the end of the reaction, the protein is excised from the resin and at the same time, the protecting groups are removed. Then, intramolecular disulfide bond-forming reaction is performed in a highly diluted solution to obtain the objective protein or its amides.

For condensation of the protected amino acids described above, a variety of activation reagents for protein synthesis may be used, but carbodiimides are particularly preferably employed. Examples of such carbodiimides include DCC, N,N′-diisopropylcarbodiimide, N-ethyl-N′-(3-dimethylaminopropyl)carbodiimide, etc. For activation by these reagents, the protected amino acids in combination with a racemization inhibitor (e.g., HOBt, HOOBt) are added directly to the resin, or the protected amino acids are previously activated in the form of symmetric acid anhydrides, HOBt esters or HOOBt esters, followed by adding the thus activated protected amino acids to the resin.

Solvents suitable for use to activate the protected amino acids or condense with the resin may be chosen from solvents that are known to be usable for protein condensation reactions. Examples of such solvents are acid amides such as N,N-dimethylformamide, N,N-dimethylacetamide, N-methylpyrrolidone, etc.; halogenated hydrocarbons such as methylene chloride, chloroform, etc.; alcohols such as trifluoroethanol, etc.; sulfoxides such as dimethylsulfoxide, etc.; ethers such as pyridine, dioxane, tetrahydrofuran, etc.; nitriles such as acetonitrile, propionitrile, etc.; esters such as methyl acetate, ethyl acetate, etc.; and appropriate mixtures of these solvents. The reaction temperature is appropriately chosen from the range known to be applicable to protein bond-forming reactions and is usually selected in the range of approximately −20° C. to 50° C. The activated amino acid derivatives are used generally in an excess of 1.5 to 4 times. The condensation is examined using the ninhydrin reaction; when the condensation is insufficient, the condensation can be completed by repeating the condensation reaction without removal of the protecting groups. When the condensation is yet insufficient even after repeating the reaction, unreacted amino acids are acetylated with acetic anhydride or acetylimidazole to cancel any possible adverse affect on the subsequent reaction.

Examples of the protecting groups used to protect the starting amino groups include Z, Boc, t-pentyloxycarbonyl, isobomyloxycarbonyl, 4-methoxybenzyloxycarbonyl, Cl—Z, Br—Z, adamantyloxycarbonyl, trifluoroacetyl, phthaloyl, formyl, 2-nitrophenylsulphenyl, diphenylphosphinothioyl, Fmoc, etc.

A carboxyl group can be protected by, e.g., alkyl esterification (in the form of linear, branched or cyclic alkyl esters of the alkyl moiety such as methyl, ethyl, propyl, butyl, t-butyl, cyclopentyl, cyclohexyl, cycloheptyl, cyclooctyl, 2-adamantyl, etc.), aralkyl esterification (e.g., esterification in the form of benzyl ester, 4-nitrobenzyl ester, 4-methoxybenzyl ester, 4-chlorobenzyl ester, benzhydryl ester, etc.), phenacyl esterification, benzyloxycarbonyl hydrazidation, t-butoxycarbonyl hydrazidation, trityl hydrazidation, or the like.

The hydroxyl group of serine can be protected through, for example, its esterification or etherification. Examples of groups appropriately used for the esterification include a lower alkanoyl group, such as acetyl group, an aroyl group such as benzoyl group, and a group derived from carbonic acid such as benzyloxycarbonyl group and ethoxycarbonyl group. Examples of a group appropriately used for the etherification include benzyl group, tetrahydropyranyl group, t-butyl group, etc.

Examples of groups for protecting the phenolic hydroxyl group of tyrosine include Bzl, Cl₂-Bzl, 2-nitrobenzyl, Br-Z, t-butyl, etc.

Examples of groups used to protect the imidazole moiety of histidine include Tos, 4-methoxy-2,3,6-trimethylbenzenesulfonyl, DNP, benzyloxymethyl, Bum, Boc, Trt, Fmoc, etc.

Examples of the activated carboxyl groups in the starting amino acids include the corresponding acid anhydrides, azides, activated esters (esters with alcohols (e.g., pentachlorophenol, 2,4,5-trichlorophenol, 2,4-dinitrophenol, cyanomethyl alcohol, p-nitrophenol, HONB, N-hydroxysuccimide, N-hydroxyphthalimide, HOBt)). As the activated amino acids in which the amino groups are activated in the starting material, the corresponding phosphoric amides are employed.

To eliminate (split off) the protecting groups, there are used catalytic reduction under hydrogen gas flow in the presence of a catalyst such as Pd-black or Pd-carbon; an acid treatment with anhydrous hydrogen fluoride, methanesulfonic acid, trifluoromethanesulfonic acid or trifluoroacetic acid, or a mixture solution of these acids; a treatment with a base such as diisopropylethylamine, triethylamine, piperidine or piperazine; and reduction with sodium in liquid ammonia. The elimination of the protecting group by the acid treatment described above is carried out generally at a temperature of approximately −20° C. to 40° C. In the acid treatment, it is efficient to add a cation scavenger such as anisole, phenol, thioanisole, m-cresol, p-cresol, dimethylsulfide, 1,4-butanedithiol or 1,2-ethanedithiol. Furthermore, 2,4-dinitrophenyl group known as the protecting group for the imidazole of histidine is removed by a treatment with thiophenol. Formyl group used as the protecting group of the indole of tryptophan is eliminated by the aforesaid acid treatment in the presence of 1,2-ethanedithiol or 1,4-butanedithiol, as well as by a treatment with an alkali such as a dilute sodium hydroxide solution and dilute ammonia.

Protection of functional groups that should not be involved in the reaction of the starting materials, protecting groups, elimination of the protecting groups and activation of functional groups involved in the reaction may be appropriately selected from publicly known groups and publicly known means.

In another method for obtaining the amides of the protein, for example, the α-carboxyl group of the carboxy terminal amino acid is first protected by amidation; the peptide (protein) chain is then extended from the amino group side to a desired length. Thereafter, a protein in which only the protecting group of the N-terminal α-amino group has been eliminated from the protein and a protein in which only the protecting group of the C-terminal carboxyl group has been eliminated are manufactured. The two proteins are condensed in a mixture of the solvents described above. The details of the condensation reaction are the same as described above. After the protected protein obtained by the condensation is purified, all the protecting groups are eliminated by the method described above to give the desired crude protein. This crude protein is purified by various known purification means. Lyophilization of the major fraction gives the amide of the desired protein.

To prepare the esterified protein, for example, the a-carboxyl group of the carboxy terminal amino acid is condensed with a desired alcohol to prepare the amino acid ester, which is followed by procedure similar to the preparation of the amidated protein above to give the desired esterified protein.

The receptor protein of the present invention or the partial peptide of the present invention (hereinafter “the partial peptide of the present invention, its amides or esters, or salts thereof” are sometimes merely referred to as “the partial peptide of the present invention” and “the receptor protein of the present invention or salts thereof” are sometimes merely referred to as “the receptor protein of the present invention”) can be manufactured by publicly known methods for peptide synthesis, or by cleaving the receptor protein of the present invention or a protein containing the receptor protein of the present invention with an appropriate peptidase. For the peptide synthesis, for example, either solid phase synthesis or liquid phase synthesis may be used. That is, the partial peptide or amino acids that can construct the receptor protein of the present invention or the partial peptide of the present invention are condensed with the remaining part. Where the product contains protecting groups, these protecting groups are removed to give the desired peptide. Publicly known methods for condensation and elimination of the protecting groups are described in 1)-5) below.

1) M. Bodanszky & M. A. Ondetti: Peptide Synthesis, Interscience Publishers, New York (1966)

2) Schroeder & Luebke: The Peptide, Academic Press, New York (1965)

3) Nobuo Izumiya, et al.: Peptide Gosei-no-Kiso to Jikken (Basics and experiments of peptide synthesis), published by Maruzen Co. (1975)

4) Haruaki Yajima & Shunpei Sakakibara: Seikagaku Jikken Koza (Biochemical Experiment) 1, Tanpakushitsu no Kagaku (Chemistry of Proteins) IV, 205 (1977)

5) Haruaki Yajima ed.: Zoku Iyakuhin no Kaihatsu (A sequel to Development of Pharmaceuticals), Vol. 14, Peptide Synthesis, published by Hirokawa Shoten

After completion of the reaction, the product may be purified and isolated by a combination of conventional purification methods such as solvent extraction, distillation, column chromatography, liquid chromatography, recrystallization, etc. to give the receptor protein of the present invention or the partial peptide of the present invention. When the receptor protein of the present invention or the partial peptide of the present invention obtained by the above methods is in a free form, they may be converted into an appropriate salt by a publicly known method; when they are obtained in a salt form, they may be converted into a free form by a publicly known method.

For the polynucleotide encoding the receptor protein of the present invention, any polynucleotide containing the base sequence (DNA or RNA, preferably DNA) encoding the receptor protein of the present invention described above can be used. Said polynucleotide may be a DNA or an RNA including mRNA encoding the receptor protein of the present invention, and it may be either double-stranded or single-stranded. When it is double-stranded, the polynucleotide may be double-stranded DNA, double-stranded RNA, or DNA:RNA hybrid. When the polynucleotide is single-stranded, it may be a sense strand (i.e., a coding strand) or an antisense strand (i.e., a non-coding strand).

Using the polynucleotide encoding the receptor protein of the present invention, mRNA of the receptor protein of the present invention can be quantified by, for example, the known method published in separate volume of Jikken Igaku 15 (7), “New PCR and its application” (1997), etc., or its modifications.

The DNA encoding the receptor protein of the present invention may be any one of genomic DNA, genomic DNA library, cDNA derived from the cells or tissues described above, cDNA library derived from the cells or tissues described above and synthetic DNA. The vector to be used for the library may be any of bacteriophage, plasmid, cosmid, phagemid and the like. In addition, the DNA can be directly amplified by reverse transcriptase polymerase chain reaction (hereinafter abbreviated as RT-PCR) with total RNA or mRNA fraction prepared from the above-described cells or tissues.

Specifically, the DNA encoding the receptor protein of the present invention may be any one of, for example, a DNA containing the base sequence represented by SEQ ID NO:2 or SEQ ID NO:6, or any DNA having a base sequence hybridizable to the base sequence represented by SEQ ID NO:2 or SEQ ID NO:6 under high stringent conditions and encoding a receptor protein which has the activities substantially equivalent to those of the receptor protein of the present invention (e.g., a cell stimulating activity, a signal transduction activity, etc.).

Specific examples of the DNA that is hybridizable to the base sequence represented by SEQ ID NO:2 or SEQ ID NO:6 under high stringent conditions include a DNA having at least about 70% homology, preferably at least about 80% homology, more preferably at least about 90% homology and most preferably at least about 95% homology, to the base sequence represented by SEQ ID NO:2 or SEQ ID NO:6.

The hybridization can be carried out by publicly known methods or by a modification thereof, for example, according to the method described in Molecular Cloning, 2nd Ed., J. Sambrook et al., Cold Spring Harbor Lab. Press, 1989. A commercially available library may also be used according to the instructions of the attached manufacturer's protocol. The hybridization can be carried out preferably under high stringent conditions.

The high stringent conditions used herein are, for example, those in a sodium concentration at about 19 mM to about 40 mM, preferably about 19 mM to about 20 mM at a temperature of about 50° C. to about 70° C., preferably about 60° C. to about 65° C. In particular, hybridization conditions in a sodium concentration at about 19 mM at a temperature of about 65° C. are most preferred.

More specifically, a DNA having the base sequence represented by SEQ ID NO: 2 may be used for the DNA encoding the receptor protein having the amino acid sequence represented by SEQ ID NO: 1, and a DNA having the base sequence represented by SEQ ID NO: 6 may be used for the DNA encoding the receptor protein having the amino acid sequence represented by SEQ ID NO: 5.

The polynucleotide containing a part of the base sequence of DNA encoding the receptor protein of the present invention or a part of the base sequence complementary to the DNA is intended to include not only a DNA encoding the partial peptide of the present invention described below but also RNA.

According to the present invention, antisense polynucleotides (nucleic acids) that can inhibit the replication or expression of the receptor protein gene of the present invention can be designed and synthesized based on the cloned or determined base sequence information of the DNA encoding the receptor protein of the present invention. Such polynucleotides (nucleic acids) are hybridizable to RNA of the receptor protein gene of the present invention and are capable of inhibiting the synthesis or function of the RNA, or are capable of regulating/controlling the expression of the receptor protein gene of the present invention through the interaction with RNAs associated with the receptor protein of the present invention. Polynucleotides complementary to the specified sequences of RNA associated with the receptor protein of the present invention and polynucleotides specifically hybridizable to RNAs associated with the receptor protein of the present invention are useful in regulating/controlling the expression of the receptor protein gene of the present invention both in vivo and in vitro. These polynucleotides are also useful for the treatment or diagnosis of diseases, etc. The term “correspond” is used to mean homologous or complementary to a specific sequence of nucleotides, base sequences or nucleic acids including the gene. As between nucleotides, base sequences or nucleic acids and peptides (proteins), the term “corresponding” usually refers to amino acids of a peptide (protein) that is instructed to be derived from the sequence of nucleotides (nucleic acids) or its complements. The 5′ end hairpin loop, 5′ end 6-base-pair repeats, 5′ end untranslated region, polypeptide translation initiation codon, protein coding region, ORF translation initiation codon, 3′ untranslated region, 3′ end palindrome region, and 3′ end hairpin loop of the receptor protein gene of the present invention may be selected as preferred target regions, though any region may be a target in the G protein-coupled receptor protein genes.

The relationship between the targeted nucleic acids and the polynucleotides complementary to at least a portion of the target, specifically the relationship between the target and the polynucleotides hybridizable to the target, may be denoted to be “antisense”. The antisense polynucleotides may be polydeoxynucleotides containing 2-deoxy-D-ribose, polydeoxynucleotides containing D-ribose, any other type of polynucleotides which are N-glycosides of a purine or pyrimidine base, or other polymers containing non-nucleotide backbones (e.g., protein nucleic acids and synthetic sequence-specific nucleic acid polymers commercially available) or other polymers containing nonstandard linkages (provided that the polymers contain nucleotides with such a configuration that allows base pairing or base stacking, as is found in DNAs or RNAs), or the like. These polynucleotides may be double-stranded DNAs, single-stranded DNAs, single-stranded RNAs or DNA:RNA hybrids, and further includes unmodified polynucleotides (or unmodified oligonucleotides), those with publicly known types of modifications, for example, those with labels known in the art, those with caps, methylated polynucleotides, those with substitution of one or more of naturally occurring nucleotides with their analogue, those with intramolecular modifications of nucleotides such as those with uncharged linkages (e.g., methyl phosphonates, phosphotriesters, phosphoramidates, carbamates, etc.), those with charged linkages or sulfur-containing linkages (e.g., phosphorothioates, phosphorodithioates, etc.), those having side chain groups such as proteins (nucleases, nuclease inhibitors, toxins, antibodies, signal peptides, poly-L-lysine, etc.) or saccharides (e.g., monosaccharides, etc.), those with intercalators (e.g., acridine, psoralen, etc.), those containing chelators (e.g., metals, radioactive metals, boron, oxidative metals, etc.), those containing alkylating agents, or those with modified linkages (e.g., a anomeric nucleic acids, etc.). Herein the terms “nucleoside”, “nucleotide” and “nucleic acid” are used to mean moieties that may contain not only the purine and pyrimidine bases, but also other heterocyclic bases, which have been modified. Such modifications may include methylated purines and pyrimidines, acylated purines and pyrimidines and other heterocyclic rings. Modified nucleosides and nucleotides also include modifications on the sugar moiety, for example, wherein one or more hydroxyl groups may optionally be replaced with a halogen, aliphatic groups, or may be converted into the corresponding functional groups such as ethers, amines, or the like.

The antisense polynucleotide (nucleic acid) of the present invention is RNA, DNA or a modified nucleic acid (RNA, DNA). Specific examples of the modified nucleic acid are, but not limited to, sulfurized and thiophosphate derivatives of nucleic acids and those resistant to degradation of polynucleoside or oligonucleoside amides. The antisense nucleic acids of the present invention can be modified preferably based on the following design, that is, by increasing the intracellular stability of the antisense nucleic acid, increasing the cell permeability of the antisense nucleic acid, increasing the affinity of the nucleic acid to the target sense strand to a higher level, or minimizing the toxicity, if any, of the antisense nucleic acid.

Many such modifications are known in the art, as disclosed in J. Kawakami et al., Pharm. Tech. Japan, Vol. 8, pp. 247, 1992; Vol. 8, pp. 395, 1992; S. T. Crooke et al. ed., Antisense Research and Applications, CRC Press, 1993; etc.

The antisense nucleic acid of the present invention may contain altered or modified sugars, bases or linkages. The antisense nucleic acid may also be provided in a specialized form such as liposomes or microspheres, or may be applied to gene therapy or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. Preferred examples of the lipids to be attached are cholesterols or derivatives thereof (e.g., cholesteryl chloroformate, cholic acid, etc.). These moieties may be attached at the 3′ end or 5′ end of the nucleic acid and may be also attached through a base, sugar, or intramolecular nucleoside linkage. Other moieties may be capping groups specifically placed at the 3′ or 5′ ends of the nucleic acid to prevent degradation by nuclease such as exonuclease, RNase, etc. Such capping groups include, but are not limited to, hydroxyl protecting groups known in the art, including glycols such as polyethylene glycol, tetraethylene glycol and the like.

The inhibitory activity of the antisense nucleic acid can be examined using the transformant of the present invention, the gene expression system of the present invention in vitro and in vivo, or the translation system of the receptor protein of the present invention in vitro and in vivo.

The DNA encoding the partial peptide of the present invention may be any DNA so long as it contains the base sequence encoding the partial peptide of the present invention described above. The DNA may also be any of genomic DNA, genomic DNA library, cDNA derived from the cells/tissues described above, cDNA library derived from the cells/tissues described above and synthetic DNA. The vector to be used for the library may be any of bacteriophage, plasmid, cosmid and phagemid. The DNA may be directly amplified by reverse transcriptase polymerase chain reaction (hereinafter simply referred to as RT-RCR) using mRNA fraction prepared from the cells/tissues described above.

Specific examples of the DNA encoding the partial peptide of the present invention include (1) a DNA that has a part of the base sequence of the DNA containing the base sequence represented by SEQ ID NO: 2 or SEQ ID NO: 6, or (2) a DNA having a base sequence hybridizable to the base sequence represented by SEQ ID NO: 2 or SEQ ID NO: 6 under high stringent conditions and containing a part of the base sequence of the DNA encoding a receptor protein having activities substantially equivalent (i.e., a ligand binding activity, a signal transduction activity, etc.) to those of the receptor protein peptide of the present invention.

Examples of the DNA that is hybridizable to the base sequence represented by SEQ ID NO: 2 or SEQ ID NO: 6 include a DNA containing the base sequence having at least about 70% homology, preferably at least about 80% homology, more preferably at least about 90% homology, most preferably at least about 95% homology, to the base sequence represented by SEQ ID NO: 2 or SEQ ID NO: 6.

For cloning of the DNA that completely encodes the receptor protein of the present invention or its partial peptides (hereinafter sometimes simply referred to as the receptor protein of the present invention), the DNA may be either amplified by PCR using synthetic DNA primers containing a part of the base sequence of the receptor protein of the present invention, or the DNA inserted into an appropriate vector can be selected by hybridization to a labeled DNA fragment or synthetic DNA that encodes a part or full region of the receptor protein of the present invention. Hybridization can be carried out, for example, in accordance with the method described in Molecular Cloning, 2nd (J. Sambrook et al., Cold Spring Harbor Lab. Press, 1989). The hybridization may also be performed using a commercially available library in accordance with the protocol described in the attached instructions.

Conversion of the base sequence of DNA can be made in accordance with publicly known methods such as the ODA-LA PCR method, the Gapped duplex method, the Kunkel method, etc., or its modifications, by using a publicly known kit available as Mutan™-super Express Km (manufactured by Takara Shuzo Co., Ltd., trademark), Mutan™-K (manufactured by Takara Shuzo Co., Ltd., trademark), or the like.

The cloned DNA encoding the receptor protein can be used as it is, depending upon purpose or, if desired, after digestion with a restriction enzyme or after addition of a linker thereto. The DNA may contain ATG as a translation initiation codon at the 5′ end thereof and TAA, TGA or TAG as a translation termination codon at the 3′ end thereof. These translation initiation and termination codons may also be added by using an appropriate synthetic DNA adapter.

The expression vector for the receptor protein of the present invention can be manufactured, for example, by (a) excising the desired DNA fragment from the DNA encoding the receptor protein of the present invention, (b) followed by ligation of the DNA fragment to an appropriate expression vector downstream a promoter in the vector.

As the vector, there may be employed plasmids derived form E. coli (e.g., pBR322, pBR325, pUC12, pUC13), plasmids derived from Bacillus subtilis (e.g., pUB110, pTP5, pC194), plasmids derived from yeast (e.g., pSH19, pSH15), bacteriophages such as λ phage, etc., animal viruses such as retrovirus, vaccinia virus, baculovirus, etc. as well as pA1-11, pXT1, pRc/CMV, pRc/RSV, pcDNAI/Neo, etc.

The promoter used in the present invention may be any promoter if it matches well with a host to be used for gene expression. In the case of using animal cells as the host, examples of the promoter include SRα promoter, SV40 promoter, LTR promoter, CMV promoter, HSV-TK promoter, etc.

Among them, CMV promoter or SRα promoter is preferably used. Where the host is bacteria of the genus Escherichia, preferred examples of the promoter include trp promoter, lac promoter, recA promoter, λP_{L} promoter, 1 pp promoter, etc. In the case of using bacteria of the genus Bacillus as the host, preferred example of the promoter are SPO1 promoter, SPO2 promoter, penP promoter, etc. In the case of using yeast as the host, preferred examples of the promoter are PHO5 promoter, PGK promoter, GAP promoter, ADH promoter, etc. In the case of using insect cells as the host, preferred examples of the promoter include polyhedrin prompter, P10 promoter, etc.

In addition to the foregoing examples, the expression vector may further optionally contain an enhancer, a splicing signal, a poly A addition signal, a selection marker, SV40 replication origin (hereinafter sometimes abbreviated as SV40ori), etc. Examples of the selection marker include dihydrofolate reductase (hereinafter sometimes abbreviated as dhfr) gene [methotrexate (MTX) resistance], ampicillin resistant gene (hereinafter sometimes abbreviated as Amp^{r}), neomycin resistant gene (hereinafter sometimes abbreviated as Neo^{r}, G418 resistance), etc. Especially when CHO (dhfr⁻) cells are used together with dhfr gene as the selection marker, selection can also be made by using a thymidine free medium.

If necessary, a signal sequence that matches with a host is added to the N-terminus of the receptor protein of the present invention. Examples of the signal sequence that can be used are Pho A signal sequence, OmpA signal sequence, etc. in the case of using bacteria of the genus Escherichia as the host; α-amylase signal sequence, subtilisin signal sequence, etc. in the case of using bacteria of the genus Bacillus as the host; MFα signal sequence, SUC2 signal sequence, etc. in the case of using yeast as the host; and insulin signal sequence, α-interferon signal sequence, antibody molecule signal sequence, etc. in the case of using animal cells as the host, respectively.

Using the vector containing the DNA encoding the receptor protein of the present invention thus constructed, transformants can be manufactured.

As the host, there may be employed, for example, bacteria belonging to the genus Escherichia, bacteria belonging to the genus Bacillus, yeast, insect cells, insects, animal cells, etc.

Specific examples of bacteria belonging to the genus Escherichia include Escherichia coli K12 DH1 [Proc. Natl. Acad. Sci. U.S.A., 60, 160 (1968)], JM103 [Nucleic Acids Research, 9, 309 (1981)], JA221 [Journal of Molecular Biology, 120, 517 (1978)], HB101 [Journal of Molecular Biology, 41, 459 (1969)], C600 [Genetics, 39, 440 (1954)], etc.

Examples of bacteria belonging to the genus Bacillus include Bacillus subtilis MI114 [Gene, 24, 255 (1983)], 207-21 [Journal of Biochemistry, 95, 87 (1984)], etc.

Examples of yeast include Saccharomyces cereviseae AH22, AH22R⁻, NA87-11A, DKD-5D, 20B-12, Schizosaccharomyces pombe NCYC1913, NCYC2036, Pichia pastoris KM71, etc.

Examples of insect cells include, for the virus AcNPV, Spodoptera frugiperda cell (Sf cell), MG1 cell derived from mid-intestine of Trichoplusia ni, High Five™ cell derived from egg of Trichoplusia ni, cells derived from Mamestra brassicae, cells derived from Estigmena acrea, etc.; and for the virus BmNPV, Bombyx mori N cell (BmN cell), etc. Examples of the Sf cell which can be used are Sf9 cell (ATCC CRL1711), Sf21 cell (both cells are described in Vaughn, J. L. et al., In Vivo, 13, 213-217 (1977)), etc.

As the insect, for example, a larva of Bombyx mori and the like can be used (Maeda et al., Nature, 315, 592 (1985)).

Examples of animal cells include monkey cell COS-7, Vero, Chinese hamster cell CHO (hereinafter referred to as CHO cell), dhfr gene deficient Chinese hamster cell CHO (hereinafter simply referred to as CHO(dhfr⁻) cell), mouse L cell, mouse AtT-20, mouse myeloma cell, rat GH 3, human FL cell, etc.

Bacteria belonging to the genus Escherichia can be transformed, for example, according to the method described in Proc. Natl. Acad. Sci. U.S.A., 69, 2110 (1972) or Gene, 17, 107 (1982).

Bacteria belonging to the genus Bacillus can be transformed, for example, according to the method described in Molecular & General Genetics, 168, 111 (1979), etc.

Yeast can be transformed, for example, according to the method described in Methods in Enzymology, 194, 182-187 (1991), Proc. Natl. Acad. Sci. U.S.A., 75, 1929 (1978), etc.

Insect cells or insects can be transformed, for example, according to the method described in Bio/Technology, 6, 47-55(1988), etc.

Animal cells can be transformed, for example, according to the method described in Saibo Kogaku (Cell Engineering), extra issue 8, Shin Saibo Kogaku Jikken Protocol (New Cell Engineering Experimental Protocol), 263-267 (1995), published by Shujunsha, or Virology, 52, 456 (1973).

Thus, the transformant transformed with the expression vector containing the DNA encoding the receptor protein can be obtained.

Where the host is bacteria belonging to the genus Escherichia or the genus Bacillus, the transformant can be appropriately incubated in a liquid medium which contains materials required for growth of the transformant such as carbon sources, nitrogen sources, inorganic materials, etc. Examples of the carbon sources include glucose, dextrin, soluble starch, sucrose, etc. Examples of the nitrogen sources include inorganic or organic materials such as ammonium salts, nitrate salts, corn steep liquor, peptone, casein, meat extract, soybean cake, potato extract, etc. Examples of the inorganic materials are calcium chloride, sodium dihydrogenphosphate, magnesium chloride, etc. In addition, yeast extracts, vitamins, growth promoting factors etc. may also be added to the medium. Preferably, pH of the medium is adjusted to about 5 to about 8.

A preferred example of the medium for incubation of the bacteria belonging to the genus Escherichia is M9 medium supplemented with glucose and Casamino acids [Miller, Journal of Experiments in Molecular Genetics, 431-433, Cold Spring Harbor Laboratory, New York, 1972]. If necessary, a chemical such as 3β-indolylacrylic acid can be added to the medium thereby to activate the promoter efficiently. Where the bacteria belonging to the genus Escherichia are used as the host, the transformant is usually cultivated at about 15° C. to about 43° C. for about 3 hours to about 24 hours. If necessary, the culture may be aerated or agitated.

Where the bacteria belonging to the genus Bacillus are used as the host, the transformant is cultivated generally at about 30° C. to about 40° C. for about 6 hours to about 24 hours. If necessary, the culture can be aerated or agitated.

Where yeast is used as the host, the transformant is cultivated in, for example, Burkholder's minimal medium [Bostian, K. L. et al., Proc. Natl. Acad. Sci. U.S.A., 77,4505 (1980)] or in SD medium supplemented with 0.5% Casamino acids [Bitter, G. A. et al., Proc. Natl. Acad. Sci. U.S.A., 81, 5330 (1984)]. Preferably, pH of the medium is adjusted to about 5 to about 8. In general, the transformant is cultivated at about 20° C. to about 35° C. for about 24 hours to about 72 hours. If necessary, the culture can be aerated or agitated.

Where insect cells or insects are used as the host, the transformant is cultivated in, for example, Grace's Insect Medium (Grace, T. C. C., Nature, 195, 788 (1962)), to which an appropriate additive such as immobilized 10% bovine serum is added. Preferably, pH of the medium is adjusted to about 6.2 to about 6.4. Normally, the transformant is cultivated at about 27° C. for about 3 days to about 5 days and, if necessary, the culture can be aerated or agitated.

Where animal cells are employed as the host, the transformant is cultivated in, for example, MEM medium containing about 5% to about 20% fetal bovine serum [Science, 122, 501 (1952)), DMEM medium (Virology, 8, 396 (1959)], RPMI 1640 medium [The Journal of the American Medical Association, 199, 519 (1967)], 199 medium [Proceeding of the Society for the Biological Medicine, 73, 1 (1950)], etc. Preferably, pH of the medium is adjusted to about 6 to about 8. The transformant is usually cultivated at about 30° C. to about 40° C. for about 15 hours to about 60 hours and, if necessary, the culture can be aerated or agitated.

As described above, the receptor protein of the present invention can be produced in the cell or on the cell membrane of the transformants, or outside the transformants.

The receptor protein of the present invention can be separated and purified from the culture described above, e.g., by the following procedures.

When the receptor protein of the present invention is extracted from the culture or cells, after cultivation, the transformant or cell is collected by a publicly known method and suspended in an appropriate buffer. The transformant or cell is then disrupted by publicly known methods such as ultrasonication, a treatment with lysozyme and/or freeze-thaw cycling, followed by centrifugation, filtration, etc. Thus, the crude extract of the receptor protein of the present invention can be obtained. The buffer may contain a protein modifier such as urea or guanidine hydrochloride, or a surfactant such as Triton X-100™, etc. When the receptor protein of the present invention is secreted in the culture broth, after completion of the cultivation the supernatant can be separated from the transformant or cell to collect the supernatant by a publicly known method.

The receptor protein of the present invention contained in the supernatant or the extract thus obtained can be purified by appropriately combining the publicly known methods for separation and purification. Such publicly known methods for separation and purification include a method utilizing difference in solubility such as salting out, solvent precipitation, etc.; a method mainly utilizing difference in molecular weight such as dialysis, ultrafiltration, gel filtration, SDS-polyacrylamide gel electrophoresis, etc.; a method utilizing difference in electric charge such as ion exchange chromatography, etc.; a method utilizing difference in specific affinity such as affinity chromatography, etc.; a method utilizing difference in hydrophobicity such as reverse phase high performance liquid chromatography, etc.; a method utilizing difference in isoelectric point such as isoelectrofocusing electrophoresis; and the like.

When the receptor protein of the present invention thus obtained is in a free form, it can be converted into the salt by publicly known methods or modifications thereof. On the other hand, when the receptor protein is obtained in the form of a salt, it can be converted into the free form or in the form of a different salt by publicly known methods or modifications thereof.

The receptor protein of the present invention produced by the recombinant can be treated, prior to or after the purification, with an appropriate protein modifying enzyme so that the protein or partial peptide can be appropriately modified to partially remove a polypeptide. Examples of the protein-modifying enzyme include trypsin, chymotrypsin, arginyl endopeptidase, protein kinase, glycosidase and the like.

The activity of the thus produced receptor protein of the present invention or salts thereof can be assayed by a binding test to a labeled ligand and by an enzyme immunoassay using the antibodies of the present invention (described below in detail).

Antibodies to the receptor protein of the present invention may be any of polyclonal and monoclonal antibodies, so long as they can recognize the receptor protein of the present invention.

The antibody to the receptor protein of the present invention can be manufactured by publicly known methods for manufacturing antibodies or antisera, using as an antigen the receptor protein of the present invention.

[Preparation of Monoclonal Antibody]

(a) Preparation of Monoclonal Antibody-Producing Cells

The receptor protein of the present invention is administered to a mammal, either solely or together with carriers or diluents to the site that can produce the antibody by the administration. In order to potentiate the antibody productivity upon the administration, complete Freund's adjuvant or incomplete Freund's adjuvant may be administered. The administration is effected usually once every 2 to 6 weeks and approximately 2 to 10 times in total. The mammals to be used include monkey, rabbit, dog, guinea pig, mouse, rat, sheep and goat, with mouse and rat being preferably used.

In the preparation of the monoclonal antibody-producing cells, animal wherein the antibody titer is noted, is selected from warm-blooded animals immunized with antigens, e.g., mice, then spleen or lymph node is collected after 2 to 5 days from the final immunization and the antibody-producing cells contained therein are fused with myeloma cells to give monoclonal antibody-producing hybridomas. The antibody titer in antisera may be determined, for example, by reacting the labeled receptor protein, which will be described later, with the antiserum followed by assaying the activity of the labeling agent bound to the antibody. The fusion may be carried out, for example, following the method of Köhler and Milstein [Nature, 256, 495 (1975)]. Examples of the fusion accelerator are polyethylene glycol (PEG), Sendai virus, etc. and PEG is preferably used.

Examples of myeloma cells include NS-1, P3U1, SP2/0, etc., with P3U1 being preferably used. A preferred ratio of the number of the antibody-producing cells (spleen cells) to the number of myeloma cells to be used ranges approximately from 1:1 to 20:1 and PEG (preferably PEG 1000 to PEG 6000) is added in a concentration of about 10 to about 80%. The cell fusion can be efficiently carried out by incubating both cells at about 20° C. to about 40° C., preferably about 30° C. to about 37° C. for about 1 minute to about 10 minutes.

Various methods can be used for screening of a monoclonal antibody-producing hybridoma. Examples of such methods include a method which comprises adding the supernatant of hybridoma to a solid phase (e.g., microplate) adsorbed with the antigen of the receptor protein of the present invention directly or together with a carrier, adding an anti-immunoglobulin antibody (when mouse cells are used for the cell fusion, anti-mouse immunoglobulin antibody is used) labeled with a radioactive substance or an enzyme or Protein A and detecting the monoclonal antibody bound to the solid phase; a method which comprises adding the supernatant of hybridoma to a solid phase adsorbed with an anti-immunoglobulin antibody or Protein A, adding the receptor protein labeled with a radioactive substance or an enzyme and detecting the monoclonal antibody bound to the solid phase, and the like.

The monoclonal antibody can be selected in accordance with publicly known methods or their modifications. In general, the selection can be effected in a medium for animal cells supplemented with HAT (hypoxanthine, aminopterin and thymidine). Any selection and growth medium may be used as far as the hybridoma can grow. For example, RPMI 1640 medium containing 1 to 20%, preferably 10 to 20% fetal bovine serum, GIT medium (Wako Pure Chemical Industries, Ltd.) containing 1 to 10% fetal bovine serum, a serum free medium for cultivation of a hybridoma (SFM-101, Nissui Seiyaku Co., Ltd.) and the like can be used for the selection and growth medium. Incubation is carried out normally at 20 to 40° C., preferably about 37° C., for 5 days to 3 weeks, preferably 1 to 2 weeks, generally in 5% CO₂. The antibody titer of the hybridoma culture supernatant can be determined as in the assay for antibody titers in antisera described above.

(b) Purification of Monoclonal Antibody

Separation and purification of a monoclonal antibody can be carried out according to the same manner as applied to conventional separation and purification for polyclonal antibodies, such as separation and purification of immunoglobulins [e.g., salting-out, alcohol precipitation, isoelectric point precipitation, electrophoresis, adsorption and desorption with ion exchangers (e.g., DEAE), ultracentrifugation, gel filtration, or a specific purification method which comprises collecting only an antibody with an activated adsorbent such as an antigen-binding solid phase, Protein A or Protein G and dissociating the binding to obtain the antibody].

[Preparation of Polyclonal Antibody]

The polyclonal antibody of the present invention can be manufactured by publicly known methods or modifications thereof. For example, a complex of immunogen (antigen of the receptor protein of the present invention) and a carrier protein is formed and a mammal is immunized with the complex in a manner similar to the method described above for the manufacture of monoclonal antibody. The product containing the antibody to the receptor protein of the present invention is collected from the immunized animal followed by separation and purification of the antibody.

In the complex of immunogen and carrier protein for immunizing mammals, the type of carrier protein and the mixing ratio of carrier to hapten may be any type and in any ratio, as long as the antibody is efficiently produced to the hapten immunized by crosslinking to the carrier. For example, bovine serum albumin, bovine thyroglobulin or keyhole limpet hemocyanin is coupled to hapten in a carrier-to-hapten weight ratio of approximately 0.1 to 20, preferably approximately 1 to 5.

A variety of condensation agents can be used for the coupling of carrier to hapten. Glutaraldehyde, carbodiimide, maleimide activated ester and activated ester reagents containing thiol group or dithiopyridyl group are used for the coupling.

The condensation product is administered to warm-blooded animals either solely or together with carriers or diluents to the site that can produce the antibody by the administration. In order to potentiate the antibody productivity upon the administration, complete Freund's adjuvant or incomplete Freund's adjuvant may be administered. The administration is usually carried out once every about 2 to about 6 weeks and about 3 to about 10 times in total.

The polyclonal antibody can be collected from the blood, ascites, etc., preferably from the blood of mammals immunized by the method described above.

The polyclonal antibody titer in antiserum can be determined by the same procedure as in the serum antibody titer described above. The polyclonal antibody can be separated and purified by the same method for separation and purification of immunoglobulin as used for the monoclonal antibody described above.

The receptor protein of the present invention or salts thereof, its partial peptide or esters, amides or salts thereof and the DNA coding therefor can be used: (1) for determination of ligand (agonist) to the receptor protein of the present invention, (2) as an agent for the prevention and/or treatment of disease associated with dysfunction of the receptor protein of the present invention, (3) as a gene diagnostic agent, (4) for a screening method of a compound or its salts that alter the expression level of the receptor protein of the present invention, (5) as an agent for the prevention and/or treatment of various diseases, comprising a compound that alters the expression level of the receptor protein of the present invention, (6) for quantification of a ligand to the receptor protein of the present invention, (7) for screening of a compound (agonist, antagonist, etc.) that alters the binding property between the receptor protein of the present invention and a ligand, (8) as an agent for the prevention and/or treatment of various diseases, comprising a compound (agonist, antagonist) that alters the binding property between the receptor protein of the present invention and a ligand, (9) for quantification of the receptor protein of the present invention, (10) for a screening method of a compound that alters the amount of the receptor protein of the present invention on a cell membrane, (11) as an agent for the

prevention and/or treatment of various diseases, comprising a compound that alters the amount of the receptor protein of the present invention on a cell membrane, (12) for neutralization of the receptor protein of the present invention by antibodies thereto, (13) for preparation of non-human animal bearing a DNA encoding the receptor protein of the present invention; and the like.

In particular, by applying the receptor binding assay system using the recombinant receptor protein expression system of the present invention, a compound (e.g., agonist, antagonist, etc.) that alters the binding property of a ligand to a G protein-coupled receptor specific to human or other mammals can be screened, and the agonist or antagonist can be used as an agent for the prevention/treatment of various diseases.

The receptor protein of the present invention, DNA encoding the receptor protein of the present invention (hereinafter sometimes collectively referred to as the DNA of the present invention) and antibodies to the receptor protein of the present invention (hereinafter sometimes referred to as the antibody of the present invention) are specifically described below, with respect to their use.

(1) Determination of a Ligand (Agonist) to the Receptor Protein of the Present Invention

The receptor protein of the present invention is useful as a reagent for searching and determining a ligand (agonist) to the receptor protein of the present invention.

That is, the present invention provides a method of determining a ligand to the receptor protein of the present invention, which comprises contacting the receptor protein of the present invention with a test compound.

Examples of test compounds include publicly known ligands (e.g., angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, vasoactive intestinal and related polypeptide (VIP), somatostatin, dopamine, motilin, amylin, bradykinin, calcitonin gene-related peptide (CGRP), leukotrienes, pancreastatin, prostaglandins, thromboxane, adenosine, adrenaline, α and β-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic polypeptide, galanin or rat cortistatin, etc.) as well as other substances, for example, tissue extracts and cell culture supernatants from mammals (e.g., human, mice, rats, swine, bovine, sheep, monkeys, etc.). For example, the tissue extract, cell culture supernatant or the like is added to the receptor protein of the present invention and fractionated while assaying the cell-stimulating activity to finally give a single ligand.

Specifically, the method of the present invention for determining a ligand comprises determining compounds (e.g., peptides, proteins, non-peptide compounds, synthetic compounds, fermentation products) or salts thereof that bind to the receptor protein of the present invention to provide the cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.), either by using the receptor protein of the present invention, or by applying the receptor (ligand) binding assay system using the constructed recombinant receptor protein expression system.

The method of the present invention for determining a ligand is characterized by, for example, measurement of the amount of a test compound bound to the receptor protein of the present invention or the cell-stimulating activity, when the receptor protein of the present invention is brought in contact with the test compound.

More specifically, the present invention provides:

(1) A method of determining a ligand to the receptor protein of the present invention, which comprises contacting a labeled test compound with the receptor protein of the present invention and measuring the amount of the labeled test compound bound to the receptor protein;

(2) A method of determining a ligand to the receptor protein of the present invention, which comprises contacting a labeled test compound with a cell containing the receptor protein of the present invention or with a membrane fraction of the cell, and measuring the amount of the labeled test compound bound to the cell or the membrane fraction;

(3) A method of determining a ligand to the receptor protein of the present invention which comprises culturing a transformant containing a DNA encoding the receptor protein of the present invention, contacting a labeled test compound with the receptor protein expressed on the cell membrane by said culturing, and measuring the amount of the labeled test compound bound to the receptor protein;

(4) A method of determining a ligand to the receptor protein of the present invention, which comprises contacting a test compound with a cell containing the receptor protein of the present invention and measuring the receptor protein-mediated cell stimulating activity (e.g., the activity that promotes or suppresses arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.); and,

(5) A method of determining a ligand to the receptor protein of the present invention, which comprises culturing a transformant containing a DNA encoding the receptor protein of the present invention, contacting a labeled test compound with the receptor protein expressed on the cell membrane through said culturing, and measuring the receptor protein-mediated cell stimulating activity (e.g., the activity that promotes or suppresses arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.).

In particular, it is preferred to perform the methods (1) to (3) thereby to confirm that a test compound can bind to the receptor protein of the present invention, followed by the methods (4) and (5) described above.

First, any protein may be usable as the receptor protein to be used for the method of determining a ligand, so long as it contains the receptor protein of the present invention described above. However, the receptor protein that is abundantly expressed using animal cells is suitable for the present invention.

The receptor protein of the present invention can be manufactured by the method for expression described above, preferably by expressing a DNA encoding the receptor protein in mammalian or insect cells. Normally DNA fragments encoding the desired portion of the protein include, but are not limited to, complementary DNAs. For example, gene fragments or synthetic DNA may also be used. In order to introduce a DNA fragment encoding the receptor protein of the present invention into host animal cells and efficiently express the same, it is preferred to incorporate the DNA fragment downstream the polyhedrin promoter of nuclear polyhedrosis virus (NPV), which is a baculovirus having insect hosts, an SV40-derived promoter, a retrovirus promoter, a metallothionein promoter, a human heat shock promoter, a cytomegalovirus promoter, an SRα promoter, or the like. The quantity and quality of the receptor expressed can be determined by a publicly known method. For example, this determination can be made by the method described in the literature [Nambi, P. et al., J. Biol. Chem., Vol. 267, pp. 19555-19559, 1992].

Accordingly, the subject containing the receptor protein of the present invention in the method of the present invention for determining a ligand may be a receptor protein purified by publicly known methods, a cell containing the receptor protein or a membrane fraction of the cell.

Where cells containing the receptor protein of the present invention are used in the method of the present invention for determining ligands, the cells may be fixed using glutaraldehyde, formalin, etc. The fixation can be made by a publicly known method.

Cells containing the receptor protein of the present invention refer to host cells that have expressed the receptor protein of the present invention, which host cells include Escherichia coli, Bacillus subtilis, yeast, insect cells, animal cells, etc.

The cell membrane fraction is a fraction abundant in cell membrane obtained by cell disruption and subsequent fractionation by a publicly known method. Useful cell disruption methods include cell squashing using a Potter-Elvehjem homogenizer, disruption using a Waring blender or Polytron (manufactured by Kinematica, Inc.), disruption by ultrasonication, disruption by cell spraying via a thin nozzle under increased pressure using a French press, and the like. Cell membrane fractionation is effected mainly by fractionation using a centrifugal force, such as centrifugation for fractionation, density gradient centrifugation, etc. For example, cell disruption fluid is centrifuged at a low rate (500 rpm to 3,000 rpm) for a short period of time (normally about 1 to 10 minutes), the resulting supernatant is then centrifuged at a higher rate (15,000 rpm to 30,000 rpm) normally for 30 minutes to 2 hours. The precipitate thus obtained is used as the membrane fraction. The membrane fraction is abundant in the receptor protein expressed and membrane components such as cell-derived phospholipids, membrane proteins, etc.

The amount of the receptor protein in the cells containing the receptor protein and in the membrane fraction is preferably 10³ to 10⁸ molecules per cell, more preferably 10⁵ to 10⁷ molecules per cell. As the amount of expression increases, the ligand binding activity per unit of the membrane fraction (specific activity) increases so that not only a highly sensitive screening system can be constructed but also large quantities of samples can be assayed with the same lot.

To perform the methods (1) through (3) for determination of a ligand to the receptor protein of the present invention, an appropriate receptor protein fraction and a labeled test compound are required.

The receptor protein fraction is preferably a fraction of naturally occurring receptor protein or a recombinant receptor fraction having an activity equivalent to that of the natural protein. Herein, the equivalent activity is intended to mean a ligand binding activity or a signal transduction activity, which is equivalent to that of natural proteins.

Preferred examples of labeled test compounds include [³H]-, [¹²⁵I]-, [¹⁴C]-, or [³⁵S]-labeled angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leukotrienes, pancreastatin, prostaglandins, thromboxane, adenosine, adrenaline, α and β-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic polypeptide, galanin or rat cortistatin, etc.

Specifically, the ligand to the receptor protein of the present invention is determined by the following procedures. First, a receptor preparation is prepared by suspending a cell containing the receptor protein of the present invention or a membrane fraction of the cell in a buffer appropriate for use in the determination method. Any buffer may be used if it does not interfere with the ligand-receptor binding, such buffers including a phosphate buffer, a Tris-HCl buffer, etc., having a pH of 4 to 10 (preferably a pH of 6 to 8). For the purpose of decreasing non-specific binding, a surfactant such as CHAPS, Tween-80™ (Kao-Atlas Inc.), digitonin, deoxycholate, etc., or various proteins such as bovine serum albumin, gelatin or the like, may optionally be added to the buffer. Further for the purpose of suppressing the degradation of the receptor or ligand by protease, a protease inhibitor such as PMSF, leupeptin, E-64 (manufactured by Peptide Institute, Inc.), pepstatin, etc. may also be added. A given amount (5,000 cpm to 500,000 cpm) of the test compound labeled with [³H], [¹²⁵I], [¹⁴C], [³⁵S] or the like is added to 0.01 ml to 10 ml of the receptor solution. To determine the amount of non-specific binding (NSB), a reaction tube containing an unlabeled test compound in a large excess is prepared as well. The reaction is carried out approximately at 0° C. to 50° C., preferably about 4° C. to about 37° C. for about 20 minutes to about 24 hours, preferably about 30 minutes to about 3 hours. After completion of the reaction, the reaction mixture is filtrated through glass fiber filter paper, etc. and rinsed with an appropriate amount of the same buffer. The residual radioactivity in the glass fiber filter paper is then measured by means of a liquid scintillation counter or a γ-counter. A test compound exceeding 0 cpm in count obtained by subtracting nonspecific binding (NSB) from the total binding (B) (B minus NSB) may be selected as a ligand (agonist) to the receptor protein of the present invention.

The method (4) or (5) above for determining a ligand to the receptor protein of the present invention can be performed as follows. The receptor protein-mediated cell-stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) can be assayed by a publicly known method, or using an assay kit commercially available. Specifically, cells containing the receptor protein are cultured on a multiwell plate, etc. Prior to the ligand determination, the medium is replaced with fresh medium or with an appropriate non-cytotoxic buffer, followed by incubation for a given period of time in the presence of a test compound, etc. Subsequently, the cells are extracted or the supernatant is recovered and the product formed is quantified by appropriate procedures. Where it is difficult to detect the production of an indicator substance for the cell-stimulating activity (e.g., arachidonic acid, etc.) due to a degrading enzyme contained in the cells, an inhibitor against such a degrading enzyme may be added prior to the assay. For detecting activities such as the cAMP production suppression, etc., the baseline production in the cells is increased by forskolin, etc. and the suppressing effect on the increased baseline production can then be detected.

The kit of the present invention for determining a ligand that binds to the receptor protein of the present invention comprises a cells containing the receptor protein of the present invention, or a membrane fraction of the cell containing the receptor protein of the present invention.

Examples of the ligand determination kit of the present invention are given below.

1. Reagents for Determining Ligands

(1) Assay and Wash Buffers

Hanks' Balanced Salt Solution (manufactured by Gibco Co.) supplemented with 0.05% bovine serum albumin (Sigma Co.).

The solution is sterilized by filtration through a 0.45 μm filter and stored at 4° C. Alternatively, the solution may be prepared at use.

(2) Receptor Protein Preparation

CHO cells on which the receptor protein of the present invention has been expressed are subcultured in a 12-well plate at the rate of 5×10⁵ cells/well and then cultured at 37° C. under 5% CO₂ and 95% air for 2 days.

(3) Labeled Test Compound

A compound labeled with commercially available [³H], [¹²⁵I], [¹⁴C], [³⁵S], etc., or a compound labeled by appropriate methods.

An aqueous solution of the compound is stored at 4° C. or −20° C. The solution is diluted to 1 μM with an assay buffer at use. A sparingly water-soluble test compound is dissolved in dimethylformamide, DMSO or methanol.

(4) Non-Labeled Compound

A non-labeled form of the same compound as the labeled compound is prepared in a concentration 100 to 1,000-fold higher than that of the labeled compound.

2. Assay Method

(1) CHO cells capable of expressing the receptor protein of the present invention are cultured in a 12-well culture plate. After washing twice with 1 ml of an assay buffer, 490 μl of the assay buffer is added to each well.

(2) After 5 μl of a labeled test compound is added, the resulting mixture is incubated at room temperature for an hour. To determine the non-specific binding, 5 μl of a non-labeled compound is added to the system.

(3) The reaction mixture is removed and the wells are washed 3 times with 1 ml of wash buffer. The labeled test compound bound to the cells is dissolved in 0.2N NaOH-1% SDS and then mixed with 4 ml of liquid scintillator A (manufactured by Wako Pure Chemical Industries, Ltd.).

(4) The radioactivity is measured using a liquid scintillation counter (manufactured by Beckman Co.).

The ligands that can bind to the receptor protein of the present invention include substances specifically present in the brain, pituitary gland, pancreas, etc. Examples of such ligands are angiotensin, bombesin, canavinoid, cholecystokinin, glutamine, serotonin, melatonin, neuropeptide Y, opioids, purines, vasopressin, oxytocin, PACAP, secretin, glucagon, calcitnonin, adrenomedulin, somatostatin, GHRH, CRF, ACTH, GRP, PTH, VIP (vasoactive intestinal and related peptide), somatostatin, dopamine, motilin, amylin, bradykinin, CGRP (calcitonin gene-related peptide), leukotriens, pancreastatin, prostaglandins, thromboxane, adenosine, adrenaline, α and β-chemokines (e.g., IL-8, GROα, GROβ, GROγ, NAP-2, ENA-78, PF4, IP10, GCP-2, MCP-1, HC14, MCP-3, I-309, MIP1α, MIP-1β, RANTES, etc.), endothelin, enterogastrin, histamine, neurotensin, TRH, pancreatic polypeptide, galanin, rat cortistatin, etc.

(2) Prophylactic and/or Therapeutic Agents for Diseases Associated with the Dysfunction of the Receptor Protein of the Present Invention

When a compound is clarified to be a ligand of the receptor protein of the present invention by the methods described in (1) above, 1) the receptor protein of the present invention, or 2) the DNA of the present invention can be used, depending on the activity possessed by the ligand, as pharmaceuticals such as agents for the prevention and/or treatment for diseases associated with the dysfunction of the receptor protein of the present invention.

For example, when the physiological activity of the ligand cannot be expected due to reduction of the receptor protein of the present invention in a patent (deficiency of the receptor protein), the activity of the ligand can be exhibited as follows: (1) the receptor protein of the present invention is administered to the patient to supplement the amount of the receptor protein; or (2) the amount of the receptor protein is increased in the patient by: i) administration of the DNA encoding the receptor protein of the present invention to express the same in the patient; or ii) incorporation of the DNA of the present invention into target cells to express the same followed by transplantation of the cells to the patient. That is, the DNA of the present invention is useful as a safe and low toxic prophylactic and/or therapeutic drug for diseases associated with dysfunction of the receptor protein of the present invention.

The receptor protein of the present invention and the DNA of the present invention are useful for the prevention and/or treatment of, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagy, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori infection, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's syndrome of bone, peptic ulcer, peripheral vessel disease, prostatic cancer, reflux esophagitis, renal insufficiency, rheumatoid arthritis, schizophrenia, sepsis, septic shock, severe systemic fungal infection, small cell lung cancer, spinal cord injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.

Where the receptor protein of the present invention is used as the prophylactic/therapeutic agent described above, the receptor protein can be prepared into a pharmaceutical preparation by a conventional means.

On the other hand, when the DNA of the present invention is used as the prophylactic/therapeutic agent described above, the DNA of the present invention may be used alone or after inserting it into an appropriate vector such as retrovirus vector, adenovirus vector or adenovirus-associated virus vector followed by a conventional means for drug administration. The DNA of the present invention may also be administered as an intact DNA, or with adjuvants to assist its uptake by a gene gun or through a catheter such as a catheter with a hydrogel.

For example, (1) the receptor protein of the present invention or (2) the DNA of the present invention can be used orally in the form of tablets which may be tablets, if necessary, coated with sugar, capsules, elixirs, microcapsules, etc., or parenterally in the form of injectable preparations such as a sterile solution or a suspension in water or with other pharmaceutically acceptable liquid. These preparations can be manufactured, e.g., by mixing (1) the receptor protein of the present invention or (2) the DNA of the present invention, with a physiologically acceptable known carrier, flavoring agent, excipient, vehicle, antiseptic, stabilizer, binder, etc., in a unit dosage form required in a generally accepted manner applied to making pharmaceutical preparations. The active ingredient in the preparation is controlled in such an amount that an appropriate dose is obtained within the specified range given.

Additives miscible with tablets, capsules, etc. include a binder such as gelatin, corn starch, tragacanth or gum arabic, an excipient such as crystalline cellulose, a swelling agent such as corn starch, gelatin, alginic acid, etc., a lubricant such as magnesium stearate, a sweetening agent such as sucrose, lactose or saccharin, a flavoring agent such as peppermint, akamono oil or cherry, and the like. When the unit dosage is in the form of capsules, liquid carriers such as oils and fats may further be used together with the additives described above. A sterile composition for injection may be formulated following a conventional manner used to make pharmaceutical compositions, e.g., by dissolving or suspending the active ingredients in a vehicle such as water for injection with a naturally occurring vegetable oil such as sesame oil, coconut oil, etc. to prepare the pharmaceutical composition. Examples of an aqueous medium for injection include physiological saline, an isotonic solution containing glucose and other auxiliary agents (e.g., D-sorbitol, D-mannitol, sodium chloride, etc.) or the like, which may be used in combination with an appropriate dissolution aid such as an alcohol (e.g., ethanol), a polyalcohol (e.g., propylene glycol, polyethylene glycol), a nonionic surfactant (e.g., polysorbate 80™ and HCO-50), etc. As an oily medium, for example, sesame oil, soybean oil or the like may be used, which can be used in combination with a dissolution aid such as benzyl benzoate, benzyl alcohol, etc.

Furthermore the prophylactic/therapeutic agent described above may also be formulated with a buffer (e.g., phosphate buffer, sodium acetate buffer), a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, etc. The thus-prepared liquid for injection is normally filled in an appropriate ampoule.

Since the thus obtained pharmaceutical preparation is safe and low toxic, the preparation can be administered to mammals (e.g., human, rat, rabbit, sheep, swine, bovine, cat, dog, monkey, etc.).

The dose of the receptor protein of the present invention varies depending on subject to be administered, target organ, symptom, route for administration, etc.; in oral administration, the dose for a patient with cancer (weighing 60 kg) is normally about 0.1 mg to about 100 mg, preferably about 1.0 to about 50 mg, and more preferably about 1.0 to about 20 mg per day. In parenteral administration, the single dose may vary depending on subject to be administered, target organ, symptom, route for administration, etc. but it is advantageous to administer the active ingredient intravenously to a patient with cancer (weighing 60 kg) in a daily dose of about 0.01 to about 30 mg, preferably about 0.1 to about 20 mg, and more preferably about 0.1 to about 10 mg. For other animal species, the corresponding dose as converted per 60 kg weight can be administered.

The dose of the DNA of the present invention varies depending on subject to be administered, target organ, symptom, route for administration, etc.; in oral administration, the dose for a patient with cancer (weighing 60 kg) is normally about 0.1 mg to about 100 mg, preferably about 1.0 to about 50 mg, and more preferably about 1.0 to about 20 mg per day. In parenteral administration, the single dose varies depending on subject to be administered, target organ, symptom, route for administration, etc. but it is advantageous to administer the active ingredient intravenously to a patient with cancer (weighing 60 kg) in a daily dose of about 0.01 to about 30 mg, preferably about 0.1 to about 20 mg, and more preferably about 0.1 to about 10 mg. For other animal species, the corresponding dose as converted per 60 kg weight can be administered.

(3) Gene Diagnostic Agent

Using the DNA of the present invention as a probe, an abnormality of the DNA or mRNA (gene abnormality) encoding the receptor protein of the present invention in mammal (e.g., human, rat, rabbit, sheep, swine, bovine, cat, dog, monkey, etc.) can be detected. Therefore, the DNA of the present invention is useful as a gene diagnostic agent for damages to the DNA or mRNA, mutation thereof, or decreased expression thereof, or increased expression or overexpression of the DNA or mRNA, or the like.

The gene diagnosis described above using the DNA of the present invention can be performed by, for example, publicly known Northern hybridization assay or PCR-SSCP assay (Genomics, 5, 874-879 (1989); Proceedings of the National Academy of Sciences of the United States of America, 86, 2766-2770 (1989)), etc.

(4) Methods of Screening Compounds that Alter the Expression Level of the Receptor Protein of the Present Invention

Using the DNA of the present invention as a probe, the DNA can be used for screening of compounds that alter the expression level of the receptor protein of the present invention.

That is, the present invention provides methods of screening compounds that alter the expression level of the receptor protein of the present invention, which comprises measuring the mRNA expression level of the receptor protein of the present invention contained in, for example, (i) (1) blood, (2) specific organs, and (3) tissues or cells isolated from the organs of, non-human mammals (e.g., rat, rabbit, sheep, swine, bovine, cat, dog, monkey, etc.) or (ii) transformants, etc.

Specifically, the mRNA levels of the receptor protein of the present invention are measured as follows.

(i) Normal or non-human animals of disease models (e.g., mice, rats, rabbits, sheep, swine, bovine, cats, dogs, monkeys, more specifically, dementia rats, obese mice, arteriosclerotic rabbits, tumor-bearing mice, etc.) receive a drug (e.g., an antidementia, hypotensive, anticancer or antiobestic drug, etc.) or physical stress (e.g., soaking stress, electric stress, light and darkness, low temperature, etc.) or the like, and the blood, specific organs (e.g., brain, liver, kidneys, etc.), or tissues or cells isolated from the organs are obtained after a specified period of time.

The mRNA of the receptor protein of the present invention contained in the cells obtained is extracted from the cells, etc., for example, in a conventional manner quantified by means of, for example, TaqManPCR, etc., and may be analyzed by the Northern blotting using publicly known methods.

(ii) Transformants expressing the receptor protein of the present invention are prepared following the methods described above, and the mRNA of the receptor protein of the present invention can be quantified and analyzed as described above.

Compounds that alter the expression level of the receptor protein of the present invention can be screened by the following procedures.

(i) To normal or disease model non-human mammals, a test compound is administered at a specified time before (30 minutes to 24 hours before, preferably 30 minutes to 12 hours before, more preferably 1 hour to 6 hours before), at a specified time after (30 minutes to 3 days after, preferably 1 hour to 2 days after, more preferably 1 hour to 24 hours after) a drug or physical stress is given, or simultaneously with a drug or physical stress. At a specified time (30 minute to 3 days, preferably 1 hour to 2 days, more preferably 1 hour to 24 hours) after administration of the test compound, the mRNA level of the receptor protein of the present invention contained in the cells are quantified and analyzed.

(ii) When transformants are cultured in a conventional manner, a test compound is mixed in the culture medium. After a specified period of time (after 1 day to 7 days, preferably after 1 day to 3 days, more preferably after 2 days to 3 days), the mRNA level of the receptor protein of the present invention contained in the transformants can be quantified and analyzed.

Compounds or salts thereof that are obtainable by the screening methods of the present invention are compounds that alter the expression level of the receptor protein of the present invention, and specifically include: (a) compounds that potentiate the cell-stimulating activity mediated by the receptor protein of the present invention (e.g., the activity that promotes or inhibits arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.) by increasing the expression level of the receptor protein; and (b) compounds that decrease the cell-stimulating activity by reducing the expression level of the receptor protein of the present invention.

The compounds may be peptides, proteins, non-peptide compounds, synthetic compounds, fermentation products, etc., and may be novel or publicly known compounds.

The compounds that potentiate the cell-stimulating activities are useful as safe and low-toxic pharmaceuticals (prophylactic and/or therapeutic agents for, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagy, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori infection, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's syndrome of bone, peptic ulcer, peripheral vessel disease, prostatic cancer, reflux esophagitis, renal insufficiency, rheumatoid arthritis, schizophrenia, sepsis, septic shock, severe systemic fungal infection, small cell lung cancer, spinal cord injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.) for potentiation of the physiological activities of the receptor protein of the present invention.

Compounds that attenuate the cell-stimulating activities are useful as safe and low-toxic pharmaceuticals for reduction of the physiological activities of the receptor protein of the present invention.

When the compound or its salts obtained by the screening methods of the present invention are used in a pharmaceutical composition, the compound can be formulated in a conventional manner. For example, as described for the pharmaceuticals containing the receptor protein of the present invention, the compound can be prepared in the form of tablets, capsules, elixir, microcapsules, sterile solutions, suspensions, etc.

Since the pharmaceutical preparations thus obtained as described above are safe and low toxic, the preparations can be administered to mammals (e.g., human, rats, rabbits, sheep, swine, bovine, cats, dogs, monkeys, etc.).

The dosage of the compound or its salts varies depending on the target individual, target organ, symptom, route for administration, etc. When it is administered orally, generally about 0.1 to about 100 mg per day, preferably about 1.0 to about 50 mg per day and more preferably about 1.0 to about 20 mg per day is administered to a patient with cancer (60 kg body weight). When it is administered parenterally, the single dose may vary depending on the target individual, target organ, symptom, route for administration, etc., but in the case of injection, it is usually desirable to intravenously inject to a patient with cancer (60 kg body weight) in a dose of about 0.01 to about 30 mg per day, preferably about 0.1 to about 20 mg per day and more preferably about 0.1 to about 10 mg per day. For other animal species, the corresponding dose as converted per 60 kg weight can be administered.

(5) Prophylactic and/or Therapeutic Drugs for Various Diseases, Containing Compounds that Alter the Expression Level of the Receptor Protein of the Present Invention

The compounds that alter the expression level of the receptor protein of the present invention can be used as prophylactic and/or therapeutic drugs for diseases associated with dysfunction of the receptor protein of the present invention.

When the compounds are used as prophylactic and/or therapeutic drugs for diseases associated with dysfunction of the receptor protein of the present invention, the compounds can be prepared into pharmaceutical preparations in a conventional manner.

For example, the compounds can be administered orally in the form of tablets, if necessary, sugar-coated tablets, capsules, elixir, microcapsules, or parenterally in the form of injection such as an sterile solution or suspension in water or other pharmaceutically acceptable liquid. For example, these preparations containing the compounds can be manufactured by mixing the compounds with physiologically acceptable known carriers, flavoring agents, fillers, vehicles, antiseptics, stabilizers, binders, etc. in a unit dosage form required for generally approved drug preparations. The amount of the active ingredient is set to give an appropriate dose within the specified range.

Additives that are miscible with tablets, capsules, etc. include, for example, a binder such as gelatin, corn starch, tragacanth or gum arabic, an excipient such as crystalline cellulose, a swelling agent such as corn starch, gelatin, alginic acid, etc., a lubricant such as magnesium stearate, a sweetening agent such as sucrose, lactose or saccharin, a flavoring agent such as peppermint, akamono oil or cherry, and the like. When the unit dosage is in the form of capsules, liquid carriers such as oils and fats may further be used together with the additives described above. A sterile composition for injection may be formulated following a conventional manner used to make pharmaceutical compositions, e.g., by dissolving or suspending the active ingredients in a vehicle such as water for injection with a naturally occurring vegetable oil such as sesame oil, coconut oil, etc. to prepare the pharmaceutical composition. Examples of an aqueous medium for injection include physiological saline, an isotonic solution containing glucose and other auxiliary agents (e.g., D-sorbitol, D-mannitol, sodium chloride, etc.) or the like, which may be used in combination with an appropriate dissolution aid such as an alcohol (e.g., ethanol), a polyalcohol (e.g., propylene glycol, polyethylene glycol), a nonionic surfactant (e.g., polysorbate 80™ and HCO-50), etc. As an oily medium, for example, sesame oil, soybean oil or the like may be used, which can be used in combination with a dissolution aid such as benzyl benzoate, benzyl alcohol, etc.

Furthermore the prophylactic/therapeutic agent described above may also be formulated with a buffer (e.g., phosphate buffer, sodium acetate buffer), a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, etc. The thus prepared liquid for injection is normally filled in an appropriate ampoule.

Since the thus obtained pharmaceutical preparation is safe and low toxic, the preparation can be administered to mammals (e.g., human, rat, rabbit, sheep, swine, bovine, cat, dog, monkey, etc.).

The dose of the receptor protein of the present invention varies depending on subject to be administered, target organ, symptom, route for administration, etc.; in oral administration, the dose for a patient with cancer (weighing 60 kg) is normally about 0.1 to 100 mg, preferably about 1.0 to 50 mg, and more preferably about 1.0 to 20 mg per day. In parenteral administration, the single dose may vary depending on subject to be administered, target organ, symptom, route for administration, etc. but it is advantageous to administer the active ingredient intravenously to a patient with cancer (weighing 60 kg) in a daily dose of about 0.01 to 30 mg, preferably about 0.1 to 20 mg, and more preferably about 0.1 to 10 mg. For other animal species, the corresponding dose as converted per 60 kg weight can be administered.

(6) Quantification of a Ligand to the Receptor Protein of the Present Invention

Since the receptor protein of the present invention has a binding property to its ligand, the ligand activity can be quantified in vivo with high sensitivity.

The method for quantification of the present invention may be performed, for example, in combination with a competitive method. Thus, a sample to be quantified is brought in contact with the receptor protein of the present invention, whereby the ligand concentration in the sample can be determined. Specifically, the quantification can be performed by the following method (1) or (2) below or its modification:

(1) Hiroshi Irie (ed.): “Radioimmunoassay” (1974, published by Kodansha, Japan); and

(2) Hiroshi Irie (ed.): “Radioimmunoassay, Second Series” (1979, published by Kodansha, Japan,).

(7) A Method of Screening a Compound (Agonist, Antagonist, etc.) that Alters the Binding Property Between the Receptor Protein of the Present Invention and its Ligand

By using the receptor protein of the present invention, or by constructing the recombinant receptor protein expression system and using the receptor-binding assay system via the expression system, the compound (e.g., peptide, protein, a non-peptide compound, a synthetic compound, fermentation product, etc.) or salts thereof that alter the binding property between the ligand and the receptor protein of the present invention can be screened efficiently.

Examples of these compounds include (a) a compound showing the cell stimulating activities mediated by the receptor protein of the present invention (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) (so-called agonists to the receptor protein of the present invention), (b) a compound having no such cell stimulating activities (so-called antagonists to the receptor protein of the present invention), (c) a compound that enhances the binding property between the ligand and the receptor protein of the present invention, or (d) a compound that decreases the binding property between the ligand and the receptor protein of the present invention (the compound (a) is screened preferably by the method of determining a ligand described above).

That is, the present invention provides a method of screening a compound or its salt that alters the binding property between the receptor protein of the present invention and the ligand, which comprises comparing the following two cases: (i) the case wherein the receptor protein of the present invention is brought in contact with the ligand; and (ii) the case wherein the receptor protein of the present invention is brought in contact with the ligand and a test compound.

According to the screening method of the present invention, the method is characterized by measuring, e.g., the amount of the ligand that binds to the receptor protein of the present invention, the cell-stimulating activities, etc. in (i) and (ii) and comparing the cases (i) and (ii).

More specifically, the present invention provides the following methods.

(1) A method of screening a compound or a salt thereof that alters the binding property between a ligand and the receptor protein of the present invention, which comprises measuring the amount of a labeled ligand bound to the receptor protein of the present invention in the case wherein the labeled ligand is brought in contact with the receptor protein of the present invention and in the case wherein the labeled ligand and a test compound are brought in contact with the receptor protein, and comparing the binding amount of the labeled ligand between the two cases.

(2) A method of screening a compound or a salt thereof that alters the binding property between a ligand and the receptor protein of the present invention, which comprises measuring the amount of a labeled ligand bound to a cell containing the receptor protein of the present invention or a membrane fraction of the cell, in the case wherein the labeled ligand is brought in contact with the cell containing the receptor protein of the present invention or the membrane fraction and in the case wherein the labeled ligand and a test compound are brought in contact with the cell containing the receptor protein or the membrane fraction, and comparing the binding amount of the labeled ligand between the two cases.

(3) A method of screening a compound or a salt thereof that alters the binding property between a ligand and the receptor protein of the present invention, which comprises measuring the amount of a labeled ligand bound to the receptor protein of the present invention, in the case wherein the labeled ligand is brought in contact with the receptor protein expressed on a cell membrane by culturing a transformant containing the DNA of the present invention and in the case wherein the labeled ligand and a test compound are brought in contact with the receptor protein expressed on the cell membrane by culturing a transformant containing the DNA of the present invention, and comparing the binding amount of the labeled ligand between the two cases.

(4) A method of screening a compound or a salt thereof that alters the binding property between a ligand and the receptor protein of the present invention, which comprises measuring the receptor-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) in the case wherein a compound (e.g., a ligand to the receptor protein of the present invention) that activates the receptor protein of the present invention is brought in contact with a cell containing the receptor protein of the present invention and in the case wherein said compound that activates the receptor protein of the present invention and a test compound are brought in contact with the cell containing the receptor protein of the present invention, and comparing the cell stimulating activities between the two cases.

(5) A method of screening a compound or a salt thereof that alters the binding property between a ligand and the receptor protein of the present invention, which comprises measuring the receptor-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) in the case wherein a compound (e.g., a ligand to the receptor protein of the present invention) that activates the receptor protein of the present invention is brought in contact with the receptor protein of the present invention expressed On a cell membrane by culturing a transformant containing the DNA of the present invention and in the case wherein said compound that activates the receptor protein of the present invention and a test compound are brought in contact with the receptor protein of the present invention expressed on a cell membrane by culturing a transformant containing the DNA of the present invention, and comparing the cell stimulating activities between the two cases.

Where G protein-coupled receptor agonists or antagonists were screened before the receptor protein of the present invention was obtained, the screening should have been made by first preparing a candidate compound using cells or tissues containing receptor proteins of rats, etc. or their cell membrane fractions (primary screening) and then confirming if the candidate compound would actually inhibit the binding between the receptor proteins and ligands (secondary screening). When the cells, tissues or cell membrane fractions are used as they are, however, other receptor proteins inevitably exist. It was thus difficult to screen agonists or antagonists to the desired receptor proteins.

However, use of the receptor protein of the present invention requires no primary screening and enables to efficiently screen the compound that inhibits the binding between a ligand and the receptor protein. Besides, it can be simply evaluated whether the compound screened is either an agonist or an antagonist.

Hereinafter the screening method of the present invention will be described more specifically.

First, the receptor protein of the present invention, which is used for the screening method of the present invention, may be any protein so long as it contains the receptor protein of the present invention described above, though membrane fractions from mammalian organs are preferably employed. Since it is very difficult to obtain human-derived organs especially, rat-derived receptor proteins, etc. expressed abundantly by use of recombinants are suitable for use in the screening.

In the manufacture of the receptor protein of the present invention, the methods described above can be used, though the DNA of the present invention is preferably expressed on mammalian cells or insect cells. As the DNA fragment encoding the target protein region, a complementary DNA may be used but is not limited thereto. For example, gene fragments or a synthetic DNA may also be used as the DNA fragment. In order to introduce the DNA fragment encoding the receptor protein of the present invention into host animal cells and express the same efficiently, the DNA fragment is preferably incorporated into a polyhedron promoter of nuclear polyhedrosis virus (NPV) belonging to the Baculovirus, an SV40-derived promoter, a promoter of retrovirus, a metallothionein promoter, a human heat shock promoter, a cytomegalovirus promoter, SRα promoter, etc. at the downstream thereof. The quantity and quality of the thus expressed receptors can be examined by a publicly known method, for example, by the method described in the literature [Nambi, P. et al., J. Biol. Chem., 267, 19555-19559, 1992].

Accordingly, in the screening method of the present invention, the substance containing the receptor protein of the present invention may be the receptor protein that is purified by publicly known methods, or a cell containing the receptor protein or a cell membrane fraction of the cell containing the receptor protein may be used as well.

Where the cell containing the receptor protein of the present invention is used in the screening method of the present invention, the cell may be fixed with glutaraldehyde, formalin, etc. The fixation may be carried out by a publicly known method.

The cell containing the receptor protein of the present invention refers to a host cell expressing the receptor protein. Examples of such a host cell include Escherichia coli, Bacillus subtilis, yeast, insect cells, animal cells, etc.

The cell membrane fraction refers to a fraction that abundantly contains cell membranes prepared by publicly known methods after disrupting cells. Examples of the cell disruption include cell squashing using a Potter-Elvehjem homogenizer, disruption using a Waring blender or Polytron (manufactured by Kinematica Inc.), disruption by ultrasonication, disruption by cell spraying via a thin nozzle under increased pressure using a French press, etc., and the like. Cell membrane fractionation is effected mainly by fractionation using a centrifugal force such as for fractionation centrifugation, density gradient centrifugation, etc. For example, cell disruption fluid is centrifuged at a low rate (500 rpm to 3,000 rpm) for a short period of time (normally about 1 minute to about 10 minutes), the resulting supernatant is then centrifuged at a higher rate (15,000 rpm to 30,000 rpm) normally for 30 minutes to 2 hours. The precipitate thus obtained is used as the membrane fraction. The membrane fraction is rich in the receptor protein expressed and membrane components such as cell-derived phospholipids, membrane proteins, or the like.

The amount of the receptor protein contained in the cells containing the receptor protein or in the membrane fraction is preferably 10³ to 10⁸ molecules per cell, more preferably 10⁵ to 10⁷ molecules per cell. As the amount of expression increases, the ligand binding activity per unit of membrane fraction (specific activity) increases so that not only the highly sensitive screening system can be constructed but also large quantities of samples can be assayed with the same lot.

To perform the methods (1) through (3) for screening the compound that alters the binding property between the ligand and the receptor protein of the present invention, an appropriate receptor protein fraction and a labeled ligand are required.

The receptor protein fraction is preferably a fraction of naturally occurring receptor protein or a recombinant receptor protein fraction having an activity equivalent to that of the naturally occurring protein. Herein, the term equivalent activity is intended to mean a ligand binding activity or a signal transduction activity that is equivalent to the activity possessed by naturally occurring receptor proteins.

Examples of the labeled ligand include ligands that are labeled with [³H], [¹²⁵I], [¹⁴C], [³⁵S], etc.

More specifically, the compound that alters the binding property between the ligand and the receptor protein of the present invention is screened by the following procedures. First, a receptor protein preparation is prepared by suspending cells containing the receptor protein of the present invention or the membrane fraction thereof in a buffer appropriate for use in the screening method. Any buffer can be used so long as it does not interfere the ligand-receptor binding, including a phosphate buffer or a Tris-HCl buffer, having pH of 4 to 10 (preferably pH of 6 to 8), etc. For the purpose of minimizing non-specific binding, a surfactant such as CHAPS, Tween-80™ (Kao-Atlas Inc.), digitonin, deoxycholate, etc., may optionally be added to the buffer. Further for the purpose of suppressing the degradation of the receptor or ligand by a protease, a protease inhibitor such as PMSF, leupeptin, E-64 (manufactured by Peptide Institute, Inc.), pepstatin, etc. may also be added. A given amount (5,000 cpm to 500,000 cpm) of the labeled ligand is added to 0.01 ml to 10 ml of the receptor solution, in which 10⁻⁴ M to 10⁻¹⁰ M of a test compound is co-present. To determine the amount of non-specific binding (NSB), a reaction tube containing an unlabeled ligand in a large excess is also provided. The reaction is carried out at approximately 0° C. to 50° C., preferably approximately 4° C. to 37° C. for about 20 minutes to about 24 hours, preferably about 30 minutes to 3 hours. After completion of the reaction, the reaction mixture is filtrated through glass fiber filter paper, etc. and washed with an appropriate volume of the same buffer. The residual radioactivity on the glass fiber filter paper is then measured by means of a liquid scintillation counter or γ-counter. When nonspecific binding (NSB) is subtracted from the count (B₀) where any antagonizing substance is absent and the resulting count (B₀ minus NSB) is made 100%, the test compound showing the specific binding amount (B minus NSB) of, e.g., 50% or less may be selected as a candidate compound.

The method (4) or (5) above for screening the compound that alters the binding property between the ligand and the receptor protein of the present invention can be performed as follows. For example, the receptor protein-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) may be determined by a publicly known method, or using an assay kit commercially available.

Specifically, the cells containing the receptor protein of the present invention are first cultured on a multiwell plate, etc. Prior to screening, the medium is replaced with fresh medium or with an appropriate non-cytotoxic buffer, followed by incubation for a given period of time in the presence of a test compound, etc. Subsequently, the cells are extracted or the supernatant is recovered and the resulting product is quantified by appropriate procedures. Where it is difficult to detect the production of the cell-stimulating activity indicator (e.g., arachidonic acid, etc.) due to a degrading enzyme contained in the cells, an inhibitor against such as a degrading enzyme may be added prior to the assay. For detecting the activities such as the cAMP production suppression activity, the baseline production in the cells is increased by forskolin or the like and the suppressing effect on the increased baseline production can be detected.

For screening through the measurement of the cell stimulating activities, cells, in which an appropriate receptor protein is expressed are necessary. Preferred cells, in which the receptor protein of the present invention is expressed are a naturally occurring cell line containing the receptor protein of the present invention and the aforesaid cell line, in which recombinant type receptor protein is expressed.

Examples of the test compounds include peptides, proteins, non-peptide compounds, synthetic compounds, fermentation products, cell extracts, plant extracts, animal tissue extracts, etc. These test compounds may be either novel or publicly known compounds.

A kit for screening the compound or a salt thereof that alters the binding property between the ligand and the receptor protein of the present invention comprises the receptor protein of the present invention, cells containing the receptor protein of the present invention, or a membrane fraction of the cells containing the receptor protein of the present invention.

Examples of the screening kit include as follows:

1. Reagent for Screening

(1) Assay and Wash Buffers

Hanks' Balanced Salt Solution (manufactured by Gibco Co.) supplemented with 0.05% bovine serum albumin (Sigma Co.).

The solution is sterilized by filtration through a 0.45 μm filter and stored at 4° C. Alternatively, the solution may be prepared at use.

(2) G Protein-Coupled Receptor Preparation

CHO cells on which the receptor protein of the present invention has been expressed are subcultured in a 12-well plate at the rate of 5×10⁵ cells/well and then cultured at 37° C. under 5% CO₂ and 95% air for 2 days.

(3) Labeled Ligand

A ligand labeled with commercially available [³H], [¹²⁵I], [¹⁴C], [³⁵S], etc. An aqueous solution of the ligand is stored at 4° C. or −20° C. The solution is diluted to 1 μM with an assay buffer at use.

(4) Standard Ligand Solution

A ligand is dissolved in PBS supplemented with 0.1% bovine serum albumin (manufactured by Sigma, Inc.) in a concentration of 1 mM, and the solution is stored at −20° C.

2. Assay Method

(1) CHO cells are cultured in a 12-well tissue culture plate to express the receptor protein of the present invention. After washing the CHO cells twice with 1 ml of the assay buffer, 490 μl of the assay buffer is added to each well.

(2) After 5 μl of a test compound solution of 10⁻³ to 10⁻¹⁰ M is added, 5 μl of the labeled ligand is added to the system followed by incubation at room temperature for an hour. To determine the amount of the non-specific binding, 5 μl of the ligand of 10⁻³ M is added, instead of the test compound.

(3) The reaction mixture is removed and washed 3 times with 1 ml each of the wash buffer. The labeled ligand bound to the cells is dissolved in 0.2N NaOH-1% SDS and mixed with 4 ml of a liquid scintillator A (manufactured by Wako Pure Chemical, Japan).

(4) Radioactivity is measured using a liquid scintillation counter (manufactured by Beckmann) and PMB (percent of the maximum binding) is calculated in accordance with the following equation 1:

PMB=[(B−NSB)/(B₀−NSB)]×100

wherein:

PMB: percent of the maximum binding

B: value when a sample is added

NSB: non-specific binding

B₀: maximum binding

The compound or a salt thereof obtainable by the screening method or the screening kit of the present invention is a compound that functions to alter the binding property between the ligand and the receptor protein of the present invention. Specifically, the compound includes (a) a compound exhibiting the cell stimulating activities mediated by the receptor protein of the present invention (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) (so-called agonists to the receptor protein of the present invention), (b) a compound exhibiting no such cell stimulating activities (so-called antagonists to the receptor protein of the present invention); or (c) a compound that decreases the binding property between the ligand and the receptor protein of the present invention.

Examples of such compounds include peptides, proteins, non-peptide compounds, synthetic compounds and fermentation products. These compounds may be either novel or publicly known compounds.

The agonist to the receptor protein of the present invention has the same physiological activity as that of the ligand to the receptor protein of the present invention. Therefore, the agonist is useful as a safe and low toxic pharmaceutical, depending upon the ligand activity (as a prophylactic and/or therapeutic agent for, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagy, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori infection, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's syndrome of bone, peptic ulcer, peripheral vessel disease, prostatic cancer, reflux esophagitis, renal insufficiency, rheumatoid arthritis, schizophrenia, sepsis, septic shock, severe systemic fungal infection, small cell lung cancer, spinal cord injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.).

The antagonist to the receptor protein of the present invention can suppress the physiological activity possessed by the ligand to the receptor protein of the present invention, and is thus useful as a safe and low toxic pharmaceutical for suppressing the ligand activity.

The compound that potentiates the binding between the ligand and the receptor protein of the present invention is useful as a safe and low toxic pharmaceutical for enhancing the physiological activity possessed by the ligand to the receptor protein of the present invention (as a prophylactic and/or therapeutic agent for, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagy, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori infection, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's syndrome of bone, peptic ulcer, peripheral vessel disease, prostatic cancer, reflux esophagitis, renal insufficiency, rheumatoid arthritis, schizophrenia, sepsis, septic shock, severe systemic fungal infection, small cell lung cancer, spinal cord injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.).

The compound that decreases the binding between the ligand and the receptor protein of the present invention is useful as a safe and low toxic pharmaceutical for decreasing the physiological activity possessed by the ligand to the receptor protein of the present invention.

When the compound or a salt thereof obtainable by the screening method or the screening kit of the present invention is used as the pharmaceutical composition described above, a conventional means may be applied to making and using the composition. For example, the compound or a salt thereof may be prepared in the form of tablets, capsules, elixir, microcapsules, sterile solutions, suspensions, etc.

Since the thus obtained pharmaceutical preparations are safe and low toxic, they can be administered to mammals (e.g., human, rat, rabbit, sheep, swine, bovine, cat, dog, monkey, etc.).

The dose of the compound or a salt thereof varies depending on subject to be administered, target organ, symptom, route for administration, etc.; in oral administration, the dose for a patient with cancer (weighing 60 kg) is normally about 0.1 to about 100 mg, preferably about 1.0 to about 50 mg and more preferably about 1.0 to about 20 mg per day. In parenteral administration, the single dose varies depending on subject to be administered, target organ, symptom, route for administration, etc. but it is advantageous to administer the active ingredient intravenously to a patient with cancer (weighing 60 kg), in a daily dose of about 0.01 to about 30 mg, preferably about 0.1 to about 20 mg, and more preferably about 0.1 to about 10 mg. For other animal species, the corresponding dose as converted per 60 kg weight can be administered.

(8) Prophylactic and/or Therapeutic Agent for Various Diseases, Comprising the Compound (Agonist, Antagonist) that Alters the Binding Property between the Receptor Protein of the Present Invention and the Ligand

The compound (agonist, antagonist) that alters the binding property between the receptor protein of the present invention and the ligand can be used as a prophylactic and/or therapeutic agent of diseases associated with dysfunction of the receptor protein of the present invention (as a prophylactic and/or therapeutic agent for, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagy, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori infection, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's syndrome of bone, peptic ulcer, peripheral vessel disease, prostatic cancer, reflux esophagitis, renal insufficiency, rheumatoid arthritis, schizophrenia, sepsis, septic shock, severe systemic fungal infection, small cell lung cancer, spinal cord injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.).

When the compound above is used as an agent for the prevention and/or treatment of diseases associated with dysfunction of the receptor protein of the present invention, a conventional means may be applied to making pharmaceutical preparations.

For example, the compound may be prepared into tablets, if necessary, coated with sugar, capsules, elixir, microcapsules, etc., for oral administration and for parenteral administration in the form of injectable preparations such as a sterile solution or suspension in water or with other pharmaceutically acceptable liquid. These preparations can be manufactured by blending the compound with a physiologically acceptable known carrier, flavoring agent, excipient, vehicle, antiseptic, stabilizer, binder, etc. in a unit dosage form required in a generally accepted manner for making pharmaceutical preparations. The active ingredient in the preparation is controlled in such a dose that an appropriate dose is obtained within the specified range given.

Additives miscible with tablets or capsules include a binder such as gelatin, corn starch, tragacanth and gum arabic, an excipient such as crystalline cellulose, a swelling agent such as corn starch, gelatin, alginic acid, etc., a lubricant such as magnesium stearate, a sweetening agent such as sucrose, lactose or saccharin, a flavoring agent such as peppermint, akamono oil or cherry, and the like. When the unit dosage is in the form of capsules, liquid carriers such as oils and fats may further be used together with the additives described above. A sterile composition for injection may be formulated by a publicly known method used to make pharmaceutical compositions, e.g., by dissolving or suspending the active ingredients in a vehicle such as water for injection with a naturally occurring vegetable oil such as sesame oil, coconut oil, etc. Examples of an aqueous medium for injection include physiological saline, an isotonic solution containing glucose and other auxiliary agents (e.g., D-sorbitol, D-mannitol, sodium chloride, etc.) or the like, and may be used in combination with an appropriate dissolution aid such as an alcohol (e.g., ethanol), a polyalcohol (e.g., propylene glycol and polyethylene glycol), a nonionic surfactant (e.g., polysorbate 80™ and HCO-50), etc. As an oily medium, for example, sesame oil, soybean oil, etc. may be used, which may be used in combination with a dissolution aid such as benzyl benzoate, benzyl alcohol, etc.

Furthermore, the prophylactic/therapeutic agent described above may also be formulated with a buffer (e.g., phosphate buffer and sodium acetate buffer) a soothing agent (e.g., benzalkonium chloride, procaine hydrochloride, etc.), a stabilizer (e.g., human serum albumin, polyethylene glycol), a preservative (e.g., benzyl alcohol, phenol, etc.), an antioxidant, and the like. The thus prepared liquid for injection is normally filled in an appropriate ampoule.

Since the thus obtained pharmaceutical preparations are safe and low toxic, they can be administered to mammals (e.g., human, rat, rabbit, sheep, swine, bovine, cat, dog, monkey, etc.).

The dose of the compound or a salt thereof varies depending on subject to be administered, target organ, symptom, route for administration, etc.; in oral administration, the dose is normally about 0.1 to about 100 mg, preferably about 1.0 to about 50 mg, more preferably about 1.0 to about 20 mg per day for a patient with cancer (weighing 60 kg). In parenteral administration, the single dose varies depending on subject to be administered, target organ, symptom, route for administration, etc. but it is advantageous to administer the active ingredient intravenously to a patient with cancer (weighing 60 kg) in a daily dose of about 0.01 to about 30 mg, preferably about 0.1 to about 20 mg, more preferably about 0.1 to about 10 mg. For other animal species, the corresponding dose as converted per 60 kg weight can be administered.

(9) Quantification of the Receptor Protein of the Present Invention

The antibody to the receptor protein of the present invention is capable of specifically recognizing the receptor protein of the present invention and thus, can be used for quantification of the receptor protein of the present invention in a test fluid, in particular, for quantification by sandwich immunoassay. That is, the present invention provides, for example, the following methods for quantification:

(i) a method of quantification of the receptor protein of the present invention in a test fluid, which comprises competitively reacting the antibody of the present invention with a test fluid and a labeled form of the receptor protein of the present invention, and measuring the ratio of the labeled receptor protein of the present invention bound to said antibody; and,

(ii) a method of quantification of the receptor protein of the present invention in a test fluid, which comprises simultaneously or continuously reacting the test fluid with the antibody of the present invention and a labeled form of the antibody of the present invention immobilized on an insoluble carrier, and measuring the activity of the labeling agent on the immobilized carrier.

In the method (ii) described above, it is preferred that one antibody is capable of recognizing the N-terminal region of the receptor protein of the present invention, while another antibody is capable of recognizing the C-terminal region of the receptor protein of the present invention.

The monoclonal antibody to the receptor protein of the present invention (hereinafter sometimes referred to as the monoclonal antibody of the present invention) may be used to assay the receptor protein of the present invention. Moreover, the receptor protein of the present invention can also be detected by means of a tissue staining, etc. For these purposes, the antibody molecule per se may be used or F(ab′)₂, Fab′ or Fab fractions of the antibody molecule may be used as well. The assay method using the antibody to the receptor protein of the present invention is not particularly limited, and any method may be used so far as it relates to a method, in which the amount of an antibody, antigen or antibody-antigen complex can be detected by a chemical or a physical means, depending on or corresponding to the amount of antigen (e.g., the amount of the receptor protein) in a test fluid to be assayed, and then calculated using a standard curve prepared by a standard solution containing the known amount of antigen. Advantageously used are, for example, nephrometry, competitive method, immunometric method and sandwich method; in terms of sensitivity and specificity, the sandwich method, which will be described later, is particularly preferred.

Examples of labeling agents, which are used for the assay method using the same, are radioisotopes, enzymes, fluorescent substances, luminescent substances, etc. Examples of radioisotopes are [¹²⁵I], [¹³¹I], [³H], [¹⁴C], etc. Preferred examples of enzymes are those that are stable and have a high specific activity, which include β-galactosidase, β-glucosidase, alkaline phosphatase, peroxidase, malate dehydrogenase, etc. Examples of fluorescent substances are fluorescamine, fluorescein isothiocyanate, etc. Examples of luminescent substances are luminol, a luminol derivative, luciferin, lucigenin, etc. Furthermore, a biotin-avidin system may be used as well for binding an antibody or antigen to a labeling agent.

In the immobilization of antigens or antibodies, physical adsorption may be used. Alternatively, chemical binding that is conventionally used for immobilization of proteins, enzymes, etc. may be used as well. Examples of the carrier include insoluble polysaccharides such as agarose, dextran, cellulose, etc.; synthetic resins such as polystyrene, polyacrylamide, silicone, etc.; or glass; and the like.

In the sandwich method, a test fluid is reacted with an immobilized form of the monoclonal antibody of the present invention (primary reaction), then reacted with a labeled form of the monoclonal antibody of the present invention (secondary reaction) and the activity of the labeling agent on the insoluble carrier is assayed; thus, the amount of the receptor protein in the test fluid can be determined. The primary and secondary reactions may be carried out in a reversed order, simultaneously or sequentially with intervals. The type of the labeling agent and the method of immobilization may be the same as those described hereinabove.

In the immunoassay by the sandwich method, it is not always necessary that the antibody used for the labeled antibody and for the solid phase should be one type or one species but a mixture of two or more antibodies may also be used for the purpose of improving the assay sensitivity, etc.

In the method of assaying the receptor protein of the present invention by the sandwich method according to the present invention, preferred monoclonal antibodies of the present invention used for the primary and the secondary reactions are antibodies, which binding sites to the receptor protein of the present invention are different from each other. Thus, the antibodies used in the primary and secondary reactions are those wherein, when the antibody used in the secondary reaction recognizes the C-terminal region of the receptor protein, the antibody recognizing the site other than the C-terminal regions, e.g., recognizing the N-terminal region, is preferably used in the primary reaction.

The monoclonal antibody of the present invention may be used in an assay system other than the sandwich method, such as the competitive method, the immunometric method or the nephrometry. In the competitive method, an antigen in a test fluid and a labeled antigen are competitively reacted with an antibody, then an unreacted labeled antigen (F) and a labeled antigen bound to the antibody (B) are separated (i.e., B/F separation) and the labeled amount of either B or F is measured to determine the amount of the antigen in the test fluid. In the reactions for such a method, there are a liquid phase method in which a soluble antibody is used as the antibody and the B/F separation is effected by polyethylene glycol, while a second antibody to the antibody is used, and a solid phase method in which an immobilized antibody is used as the first antibody or a soluble antibody is used as the first antibody, while an immobilized antibody is used as the second antibody.

In the immunometric method, an antigen in a test fluid and an immobilized antigen are competitively reacted with a given amount of a labeled antibody followed by separating the solid phase from the liquid phase; or an antigen in a test fluid and an excess amount of labeled antibody are reacted, then an immobilized antigen is added to bind an unreacted labeled antibody to the solid phase and the solid phase is separated from the liquid phase. Thereafter, the labeled amount of any of the phases is measured to determine the antigen amount in the test fluid.

In the nephrometry, the amount of insoluble sediment, which is produced as a result of the antigen-antibody reaction in a gel or in a solution, is measured. Even when the amount of an antigen in a test fluid is small and only a small amount of the sediment is obtained, a laser nephrometry utilizing laser scattering can be suitably used.

In applying each of those immunoassays to the assay method of the present invention, any special conditions, operations, etc. are not required. The assay system for the receptor protein of the present invention may be constructed in addition to conditions or operations conventionally used for each of the methods, taking technical consideration by one skilled in the art into account. For the details of such conventional technical means, a variety of reviews, reference books, etc. may be referred to [for example, Hiroshi Irie (ed.): “Radioimmunoassay” (published by Kodansha, 1974); Hiroshi Irie (ed.): “Radioimmunoassay; Second Series” (published by Kodansha, 1979); Eiji Ishikawa, et al. (ed.): “Enzyme Immunoassay” (published by Igaku Shoin, 1978); Eiji Ishikawa, et al. (ed.): “Enzyme Immunoassay” (Second Edition) (published by Igaku Shoin, 1982); Eiji Ishikawa, et al. (ed.): “Enzyme Immunoassay” (Third Edition) (published by Igaku Shoin, 1987); “Methods in Enzymology” Vol. 70 (Immuochemical Techniques (Part A)); ibid., Vol. 73 (Immunochemical Techniques (Part B)); ibid., Vol. 74 (Immunochemical Techniques (Part C)); ibid., Vol. 84 (Immunochemical Techniques (Part D: Selected Immunoassays)); ibid., Vol. 92 (Immunochemical Techniques (Part E: Monoclonal Antibodies and General Immunoassay Methods)); ibid., Vol. 121 (Immunochemical Techniques (Part I: Hybridoma Technology and Monoclonal Antibodies)) (published by Academic Press); etc.]

As described above, the receptor protein of the present invention can be quantified with high sensitivity, using the antibody of the present invention.

Furthermore, by quantifying the receptor protein of the present invention in vivo using the antibody of the present invention, diagnosis of various diseases associated with the dysfunction of the receptor protein of the present invention can be made.

The antibody to the receptor protein of the present invention can be employed to specifically detect the receptor protein of the present invention present in a test fluid such as a body fluid, tissues, etc. The antibody can also be used for the preparation of an antibody column used to purify the receptor protein of the present invention, detect the receptor protein of the present invention in each fraction upon purification, analysis of the behavior of the receptor protein of the present invention in the cells under investigation.

(10) Method of Screening Compounds that Alter the Amount of the Receptor Protein of the Present Invention in Cell Membranes

Since the antibody of the present invention specifically recognizes the receptor protein of the present invention, the antibody can be used for screening the compounds that alter the amount of the receptor protein of the present invention in cell membranes.

That is, the present invention provides, for example, the following methods:

(i) a method of screening compounds that alter the amount of the receptor protein of the present invention in cell membranes, which comprises disrupting 1) blood, 2) specific organs, or 3) tissues or cells isolated from the organs of non-human mammals, isolating cell membrane fractions and then quantifying the receptor protein of the present invention contained in the cell membrane fractions;

(ii) a method of screening compounds that alter the amount of the receptor protein of the present invention in cell membranes, which comprises disrupting transformants, etc. expressing the receptor protein of the present invention, isolating cell membrane fractions, and then quantifying the receptor protein of the present invention contained in the cell membrane fractions;

(iii) a method of screening compounds that alter the amount of the receptor protein of the present invention on cell membranes, which comprises sectioning 1) blood, 2) specified organs, 3) tissues or cells isolated from the organs of non-human mammals, immunostaining, and then quantifying the staining intensity of the receptor protein on the cell surface layer to confirm the receptor protein of the present invention on the cell membrane; and,

(iv) a method of screening compounds that alter the amount of the receptor protein of the present invention on cell membranes, which comprises sectioning transformants, etc. expressing the receptor protein of the present invention, immunostaining, and then quantifying the staining intensity of the receptor protein on the cell surface layer to confirm the receptor protein of the present invention on the cell membrane.

Specifically, the receptor protein of the present invention contained in cell membrane fractions are quantified as follows.

(i) Normal or disease model non-human mammals (e.g., mice, rats, rabbits, sheep, swine, bovine, cats, dogs, monkeys, etc., more specifically, dementia rats, obese mice, rabbits with arteriosclerosis, tumor-bearing mice, etc.) are administered with a drug (e.g., anti-dementia agents, hypotensive agents, anticancer agents, antiobestic agents, etc.), physical stress (e.g., soaking stress, electric shock, light and darkness, low temperature, etc.) or the like, and the blood, specific organs (e.g., brain, liver, kidneys, etc.), or tissue or cells isolated from the organs are obtained after a specified period of time. The obtained organs, tissues, cells, etc. are suspended in, for example, an appropriate buffer (e.g., Tris hydrochloride buffer, phosphate buffer, Hepes buffer, etc.), and the organs, tissues or cells are disrupted, and the cell membrane fraction is obtained using surfactants (e.g., Triton-X 100™, Tween 20™) and further using techniques such as centrifugal separation, filtration, column fractionation, etc.

The cell membrane fraction refers to a fraction abundant in cell membranes obtained by cell disruption and subsequent fractionation by publicly known methods. Useful cell disruption methods include cell squashing using a Potter-Elvehjem homogenizer, disruption using a Waring blender or Polytron (manufactured by Kinematica Inc.), disruption by ultrasonication, and disruption by cell spraying through thin nozzles under an increased pressure using a French press or the like. Cell membrane fractionation is effected mainly by fractionation using a centrifugal force, such as centrifugation for fractionation, density gradient centrifugation, etc. For example, cell disruption fluid is centrifuged at a low speed (500 rpm to 3,000 rpm) for a short period of time (normally about 1 to about 10 minutes), the resulting supernatant is then centrifuged at a higher speed (15,000 rpm to 30,000 rpm) normally for 30 minutes to 2 hours. The precipitate thus obtained is used as the membrane fraction. The membrane fraction is rich in the receptor protein of the present invention expressed and membrane components such as cell-derived phospholipids, membrane proteins, etc.

The receptor protein of the present invention contained in the cell membrane fraction can be quantified by, for example, the sandwich immunoassay, Western blotting analysis, etc. using the antibodies of the present invention.

The sandwich immunoassay can be performed as described above, and Western blotting can be performed by publicly known methods.

(ii) Transformants expressing the receptor protein of the present invention are prepared following the method described above, and the receptor protein of the present invention contained in cell membrane fractions can be quantified.

The compounds that alter the amount of the receptor protein of the present invention in cell membranes can be screened as follows.

(i) To normal or disease model non-human mammals, a test compound is administered at a specified time before (30 minutes to 24 hours before, preferably 30 minutes to 12 hours before, more preferably J hour to 6 hours before), at a specified time after (30 minutes to 3 days after, preferably 1 hour to 2 days after, more preferably 1 hour to 24 hours after) a drug or physical stress is given, or simultaneously with a drug or physical stress. At a specified time (30 minute to 3 days, preferably 1 hour to 2 days, more preferably 1 hour to 24 hours) after administration of the test compound, the amount of the receptor protein of the present invention on the cell membranes are quantified and analyzed.

Specifically, the receptor protein of the present invention contained in cell membrane fractions are confirmed as follows.

(iii) Normal or disease model non-human mammals (e.g., mice, rats, rabbits, sheep, swine, bovine, cats, dogs, monkeys, etc., more specifically, dementia rats, obese mice, rabbits with arteriosclerosis, tumor-bearing mice, etc.) are administered with a drug (e.g., anti-dementia agents, hypotensive agents, anticancer agents, antiobestic agents, etc.) or physical stress (e.g., soaking stress, electric shock, light and darkness, low temperature, etc.) or the like, and the blood, specific organs (e.g., brain, liver, kidneys, etc.), or tissue or cells isolated from the organs are collected after a specified period of time. Tissue sections are prepared from the thus obtained organs, tissues, cells, etc. in a conventional manner followed by immunostaining using the antibody of the present invention. By confirming the protein on the cell membranes through quantification of the staining intensity of the receptor protein on the cell surface layer, the amount of the receptor protein of the present invention or its partial peptide on the cell membranes can be confirmed quantitatively or qualitatively.

(iv) The confirmation can also be made by the similar method, using transformants expressing the receptor protein of the present invention.

The compounds or its salts, which are obtainable by the screening methods of the present invention, are the compounds that alter the amount of the receptor protein of the present invention. Specifically, these compounds are; (a) compounds that potentiate the cell-stimulating activity mediated by the receptor protein of the present invention (e.g., activity that promotes or inhibits arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.), by increasing the amount of the receptor protein of the present invention on cell membranes; and (b) compounds that lower the cell stimulating-activity by decreasing the amount of the receptor protein of the present invention on cell membranes.

The compounds may be peptides, proteins, non-peptide compounds, synthetic compounds, fermentation products, etc., and may be novel or publicly known compounds.

The compound that potentiates the cell stimulating activity is useful as a safe and low toxic pharmaceutical for enhancing the physiological activity of the receptor protein of the present invention (as a prophylactic and/or therapeutic agent for, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagy, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori infection, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's syndrome of bone, peptic ulcer, peripheral vessel disease, prostatic cancer, reflux esophagitis, renal insufficiency, rheumatoid arthritis, schizophrenia, sepsis, septic shock, severe systemic fungal infection, small cell lung cancer, spinal cord injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.).

The compound that attenuates the cell-stimulating activity is useful as a safe and low-toxic pharmaceutical for reduction of the physiological activity of the receptor protein of the present invention.

When the compound or its salts, which are obtainable by the screening methods of the present invention, are used in a pharmaceutical composition, a conventional means applies to making pharmaceuticals. For example, as described for the pharmaceuticals containing the receptor protein of the present invention, the compound can be prepared in the form of tablets, capsules, elixir, microcapsules, sterile solution, suspension, etc.

Since the pharmaceutical preparations thus obtained are safe and low-toxic, the preparations can be administered to mammals (e.g., human, rat, rabbit, sheep, swine, bovine, cat, dog, monkey, etc.).

The dose of the compound or its salts may vary depending on subject to be administered, target organ, condition, route for administration, etc.; in oral administration, e.g., to a patient with cancer (as 60 kg body weight), the dose is normally about 0.1 to about 100 mg, preferably about 1.0 to about 50 mg, and more preferably about 1.0 to about 20 mg per day. In parenteral administration, the single dose varies depending on subject to be administered, target organ, condition, route for administration, etc. but it is advantageous to administer the active ingredient intravenously, e.g., to a patient with cancer (as 60 kg body weight), in a daily dose of about 0.01 to about 30 mg, preferably about 0.1 to about 20 mg, and more preferably about 0.1 to about 10 mg. For other animal species, the corresponding dose as converted per 60 kg body weight can be administered.

(11) Prophylactic and/or Therapeutic Agent for Various Diseases, Comprising the Compound that Alters the Amount of the Receptor Protein of the Present Invention on Cell Membrane

As described above, the receptor protein of the present invention is considered to play some important role in vivo, such as a role in the central function. Therefore, the compound that alters the amount of the receptor protein of the present invention on cell membranes can be used as a prophylactic and/or therapeutic agent for diseases associated with dysfunction of the receptor protein of the present invention.

When the compound is used as a prophylactic and/or therapeutic agent for diseases associated with dysfunction of the receptor protein of the present invention, the compound can be prepared in pharmaceutical preparations in a conventional manner.

For example, the compound can be administered orally as tablets, if necessary coated with sugar, capsules, elixir, microcapsule, etc., or parenterally as injection such as an sterile solution or suspension in water or other pharmaceutically acceptable liquid. For example, pharmaceutical preparations of the compound can be manufactured by blending the compound with physiologically acceptable known carrier, flavor, filler, vehicle, antiseptic, stabilizer, binder, etc. in a unit-dosage form required for generally approved drug preparation. The amount of the active ingredient is set to give an appropriate dose within the specified range.

Additives used to be miscible with tablets, capsules, etc. include, for example, binders such as gelatin, cornstarch, tragacanth, gum arabic, etc., fillers such as crystalline cellulose, imbibers such as cornstarch, gelatin, alginic acid, etc., lubricants such as magnesium stearate, sweeteners such as sucrose, lactose or saccharin, flavors such as peppermint, akamono oil, cherry, etc. When the dosage form is in the form of capsules, liquid carrier such as fat and oil may be contained. Sterile compositions for injection can be formulated in a conventional manner to make pharmaceutical preparation, by dissolving or suspending the active ingredients in a vehicle, e.g., water for injection, naturally occurring vegetable oil such as sesame oil, coconut oil, etc. For the aqueous medium for injection, for example, physiological saline, isotonic solution (e.g., D-sorbitol, D-mannitol, sodium hydrochloride, etc.) containing glucose and other adjuvant are used. Appropriate dissolution aids, for example, an alcohol (e.g., ethanol), polyalcohol (e.g., propylene glycol, polyethylene glycol), nonionic surfactant (e.g., polysorbate 80™, HCO-50) and the like may be used in combination. For the oily medium, for example, sesame oil, soybean oil, etc. are used, and dissolution aids such as benzyl benzoate, benzyl alcohol, etc. may be used in combination therewith.

The prophylactic/therapeutic agents described above may be formulated with, e.g., buffers (e.g., phosphate buffer, sodium acetate buffer), soothing agents (e.g., benzalkonium chloride, procaine hydrochloride, etc.), stabilizers (e.g., human serum albumin, polyethylene glycol, etc.), preservatives (e.g., benzyl alcohol, phenol, etc.), antioxidants, and the like. The injection thus prepared is usually filled in appropriate ampoules.

Since the pharmaceutical preparations thus obtained are safe and low toxic, the preparations can be administered to, for example, mammals (e.g., human, rat, rabbit, sheep, swine, bovine, cat, dog, monkey, etc.).

The dose of the compound or a salt thereof varies depending on subject to be administered, target organ, symptom, route for administration, etc.; in oral administration, the dose is normally about 0.1 to about 100 mg, preferably about 1.0 to about 50 mg, more preferably about 1.0 to about 20 mg per day for a patient with cancer (as weighing 60 kg). In parenteral administration, the single dose varies depending on subject to be administered, target organ, symptom, route for administration, etc. but it is advantageous to administer the active ingredient intravenously to a patient with cancer (as weighing 60 kg) in a daily dose of about 0.01 to about 30 mg, preferably about 0.1 to about 20 mg, more preferably about 0.1 to about 10 mg. For other animal species, the corresponding dose as converted per 60 kg weight can be administered.

(12) Neutralization with the Antibody to the Receptor Protein of the Present Invention

The activity of the antibody to the receptor protein of the present invention that neutralizes the receptor protein means an activity of inactivating the function of signal transduction, in which the receptor protein of the present invention participate. Therefore, when the antibody has the neutralizing activity, the antibody can inactivate the signal transduction, in which the receptor protein participate, for example, the receptor protein-mediated cell stimulating activities (e.g., the activities that promote or suppress arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.). Thus, the antibody can be used for the prevention and/or treatment of diseases caused by overexpression of the receptor protein.

(13) Preparation of Non-Human Animals Containing the DNA Encoding the Receptor Protein of the Present Invention

Using the DNA of the present invention, transgenic non-human animals that express the receptor protein of the present invention can be prepared. Examples of the non-human animals are mammals (e.g., rats, mice, rabbits, sheep, swine, bovine, cats, dogs, monkeys, etc.) (hereinafter merely referred to as animal) can be used, with particularly preferred being mice and rats.

To transfer the DNA of the present invention to a target animal, it is generally advantageous to use the DNA as a gene construct ligated downstream a promoter capable of expressing the DNA in an animal cell. For example, when the DNA of the present invention is transferred, a gene construct wherein the DNA is ligated downstream a promoter that can expresses the DNA of the present invention derived from an animal, which is highly homologous to the DNA of the present invention, is microinjected to, e.g., fertilized ova. Thus, the DNA-transferred animal capable of producing a high level of the receptor protein of the present invention can be prepared. Examples of the promoter that can be used are a virus-derived promoter, a ubiquitous expression promoter such as metallothionein, etc. may be used but an NGF gene promoter, an enolase gene promoter, etc. that are specifically expressed in the brain are used preferably.

The transfer of the DNA of the present invention at the fertilized egg cell stage secures the presence of the DNA in all germ and somatic cells in the target animal. The presence of the receptor protein of the present invention in the germ cells in the DNA-transferred animal means that all germ and somatic cells contain the receptor protein of the present invention in all progenies of the animal. The progenies of the animal that took over the gene contain the receptor protein of the present invention in all germ and somatic cells.

The transgenic animal to which the DNA of the present invention is transferred can be subjected to mating and breeding for generations under common breeding circumstance, as the DNA-bearing animal, after confirming that the gene can be stably retained. Moreover, male and female animals having the desired DNA are mated to give a homozygote having the transduced gene in both homologous chromosomes and then the male and female animals are mated so that such breeding for generations that progenies contain the DNA can be performed.

The transgenic animal to which the DNA of the present invention has been transferred is useful as the animal for screening the agonist or antagonist to the receptor protein of the present invention, since the receptor protein of the present invention is abundantly expressed.

The transgenic animal to which the DNA of the present invention has been transferred can also be used as the cell sources for tissue culture. The receptor protein of the present invention can be analyzed by, for example, direct analysis of DNAs or RNAs in tissues of the DNA-transferred mice of the present invention, or by analysis of tissues containing the receptor protein expressed from the gene. Cells from tissues containing the receptor protein of the present invention are cultured by the standard tissue culture technique. Using these cells the function of the cells from tissues that are generally difficult to culture, for example, cells derived from the brain and peripheral tissues, can be studied. Using these cells it is possible to select pharmaceuticals, for example, that increase the function of various tissues. Where a highly expressing cell line is available, the receptor protein of the present invention can be isolated and purified from the cell line.

In the specification and drawings, the codes of bases and amino acids are denoted in accordance with the IUPAC-IUB Commission on Biochemical Nomenclature or by the common codes in the art, examples of which are shown below. For amino acids that may have the optical isomer, L form is presented unless otherwise indicated.

DNA: deoxyribonucleic acid

cDNA: complementary deoxyribonucleic acid

A: adenine

T: thymine

G: guanine

C: cytosine

RNA: ribonucleic acid

mRNA: messenger ribonucleic acid

dATP: deoxyadenosine triphosphate

dTTP: deoxythymidine triphosphate

dGTP: deoxyguanosine triphosphate

dCTP: deoxycytidine triphosphate

ATP: adenosine triphosphate

EDTA: ethylenediaminetetraacetie acid

SDS: sodium dodecyl sulfate

Gly: glycine

Ala: alanine

Val: valine

Leu: leucine

Ile: isoleucine

Ser: serine

Thr: threonine

Cys: cysteine

Met: methionine

Glu: glutamic acid

Asp: aspartic acid

Lys: lysine

Arg: arginine

His: histidine

Phe: phenylalanine

Tyr: tyrosine

Trp: tryptophan

Pro: proline

Asn: asparagine

Gln: glutamine

pGlu: pyroglutamic acid

Me: methyl group

Et: ethyl group

Bu: butyl group

Ph: phenyl group

TC: thiazolidine-4(R)-carboxamide group

Substituents, protecting groups and reagents generally used in the specification are denoted by the codes below.

Tos: p-toluenesulfonyl

CHO: formyl

Bzl: benzyl

Cl₂Bzl: 2,6-dichlorobenzyl

Bom: benzyloxymethyl

Z: benzyloxycarbonyl

Cl—Z: 2-chlorobenzyloxycarbonyl

Br—Z: 2-bromobenzyloxycarbonyl

Boc: t-butoxycarbonyl

DNP: dinitrophenol

Trt: trityl

Bum: t-butoxymethyl

Fmoc: N-9-fluorenylmethoxycarbonyl

HOBt: 1-hydroxybenztriazole

HOOBt: 3,4-dihydro-3-hydroxy-4-oxo-1,2,3-benzotriazine

HONB: 1-hydroxy-5-norbornene-2,3-dicarboximide

DCC: N,N′-dichlorohexylcarbodiimide

The sequence identification numbers in the sequence listing of the specification indicates the following sequences, respectively.

[SEQ ID NO: 1]

The amino acid sequence of mouse heart-derived novel receptor protein mML of the present invention.

[SEQ ID NO: 2]

The base sequence of cDNA encoding the mouse heart-derived novel receptor protein mML of the present invention having the amino acid sequence represented by SEQ ID NO:1.

[SEQ ID NO: 3]

The base sequence of primer 1 used in EXAMPLE 1 or 3 later described.

[SEQ ID NO: 4]

The base sequence of primer 2 used in EXAMPLE 1 or 3 later described.

[SEQ ID NO: 5]

The amino acid sequence of rat whole brain-derived novel receptor protein of the present invention.

[SEQ ID NO: 6]

The base sequence of cDNA encoding the rat whole brain-derived novel receptor protein of the present invention having the amino acid sequence represented by SEQ ID NO:1.

[SEQ ID NO: 7]

The base sequence of primer rMF2 used in EXAMPLE 2 later described.

[SEQ ID NO: 8]

The base sequence of primer rMR2 used in EXAMPLE 2 later described.

[SEQ ID NO: 9]

The amino acid sequence of rat cortistatin used in EXAMPLE 4 later described.

Transformant Escherichia coli JM109/pTAmML5 having the base sequence encoding the amino acid sequence of rat cortistatin employed in EXAMPLE 4 later described has been deposited since Nov. 9, 2000 on the Ministry of International Trade and Industry, Agency of Industrial Science and Technology, National Institute of Bioscience and Human Technology (NIBH), located at 1-1-3, Higashi, Tsukuba-shi, Ibaraki, Japan, as the Accession Number FERM BP-7357 and since Oct. 24, 2000 on Institute for Fermentation (IFO), located at 2-17-85, Juso Honcho, Yodogawa-ku, Osaka-shi, Osaka, Japan, as the Accession Number IFO 16490.

Transformant Escherichia coli JM109/pTArML1 acquired in EXAMPLE 2 later described has been deposited since Nov. 9, 2000 on the Ministry of International Trade and Industry, Agency of Industrial Science and Technology, National Institute of Bioscience and Human Technology (NIBH), located at 1-1-3, Higashi, Tsukuba-shi, Ibaraki, Japan, as the Accession Number FERM BP-7359 and since Oct. 24, 2000 on Institute for Fermentation (IFO), located at 2-17-85, Juso Honcho, Yodogawa-ku, Osaka-shi, Osaka, Japan, as the Accession Number IFO 16492.

### EXAMPLES

The present invention will be described in more detail below, with reference to EXAMPLES, but is not deemed to limit the scope of the present invention thereto. The gene manipulation procedures using Escherichia coli were performed according to the methods described in the Molecular Cloning.

### Example 1

Acquisition of Mouse Type ML Receptor Gene

For acquisition of mouse type ML receptor, the following 2 synthetic DNAs were synthesized.

| mmF: 5′-GTCGACGCCACAGAGAAAGCCATCTTCCT | (SEQ ID NO:3) |
|---|---|
| GGA-3′ | |
| mmR: 5′-GCTAGCTTCCTTGGGGATGTCCTAGCTAA | (SEQ ID NO:4) |
| AGG-3′ | |

Using these synthetic DNAs, mouse type ML receptor gene was acquired from mouse heart cDNAs by PCR. The reaction solution for PCR contained 1 μl of cDNA solution (0.1 ng poly(A)⁺ RNA-derived), 0.5 μl of mmF (10 μM), 0.5 μl of mmR2 (10 μM), 2.5 μl of 10×reaction solution attached, 2.5 μl of dNTP (10 mM) and 0.5 μl of KlenTaq (Clontech, Inc.), to which 17.5 μl of distilled water was added to make 25 μl in total. The reaction solution was subjected to PCR using Thermal Cycler 9600. PCR was conducted under the conditions of denaturation at 95° C. for 2 minutes followed by 38 repetitions of the cycle set to treat at 98° C. for 10 seconds, 65° C. for 20 seconds and 72° C. for 40 seconds. After it was confirmed by electrophoresis using an aliquot of the PCR product that the PCR product of about 1.0 kb was amplified, the PCR product was directly sequenced to obtain the sequence shown by SEQ ID NO:2. The amino acid sequence deduced from the DNA sequence of SEQ ID NO:2 was the sequence represented by SEQ ID NO:1. Homology to mouse mas receptor is shown in FIG. 1.

### Example 2

Acquisition of Rat Type ML Receptor Gene

For acquisition of rat type ML receptor, the following 2 synthetic DNAs were synthesized.

| rMF2: 5′-ATGGAGCCATTGGCAACAACCTTGTGTC | (SEQ ID NO:7) |
|---|---|
| CT-3′ | |
| rMR2: 5′-TCATAAGGGCAGGGAGAATTGTACCTCA | (SEQ ID NO:8) |
| TT-3′ | |

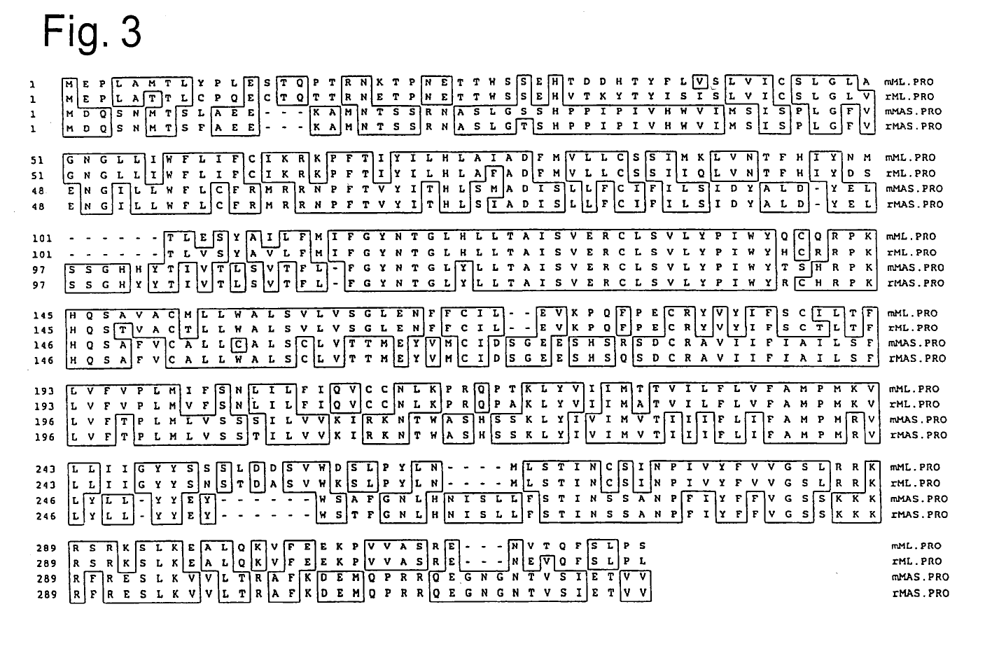

Using these synthetic DNAs, rat type ML receptor gene was acquired from rat whole brain cDNAs by PCR. The reaction solution for PCR contained 1 μl of cDNA solution (0.1 ng poly(A)⁺ RNA-derived), 0.5 μl of rMF2 (10 μM), 0.5 μl of rMR2 (10 μM), 2.5 μl of 10×reaction solution attached, 2.5 μl of dNTP (10 mM) and 0.5 μl of KlenTaq (Clontech, Inc.), to which 17.5 μl of distilled water was added to make 25 μl in total. The reaction solution was subjected to PCR using Thermal Cycler 9600. PCR was conducted under the conditions of denaturation at 95° C. for 2 minutes followed by 33 repetitions of the cycle set to treat at 98° C. for 10 seconds, 65° C. for 30 seconds and 72° C. for 60 seconds. After it was confirmed by electrophoresis using an aliquot of the PCR product that the PCR product of about 1.0 kb was amplified, the PCR product was directly sequenced to obtain the sequence shown by SEQ ID NO:6. The amino acid sequence deduced from the DNA sequence of SEQ ID NO:6 was the sequence represented by SEQ ID NO:5. The DNA acquired was prepared and transfected to pCR2.1 TOPO vector using TA cloning kit (Invitrogen) to transform E. coli JM 109. Thus, E. coli JM109/pTArML1 was obtained. Homology of rat type ML receptor to mouse type ML receptor, rat type mas receptor and mouse type mas receptor is shown in FIG. 3. The homology of rat type ML receptor to mouse type ML receptor was 89%, and the homology of rat type ML receptor to rat type mas receptor was 44%.

### Example 3

Preparation of Mouse Type Mas-Like (ML) Receptor-Expressing CHO Cells

Mouse type ML receptor was acquired as follows. Based on the sequence of mouse type ML receptor acquired in EXAMPLE 1, the following 2 synthetic DNAs were synthesized.

| mmF: 5′-GTCGACGCCACAGAGAAAGCCATCTTCCT | (SEQ ID NO:3) |
|---|---|
| GGA-3′ | |
| mmR: 5′-GCTAGCTTCCTTGGGGATGTCCTAGCTAA | (SEQ ID NO:4) |
| AGG-3′ | |

Using these synthetic DNAs, the receptor was acquired from mouse heart cDNAs by PCR. The reaction solution for PCR contained 1 μl of mouse heart cDNA solution (0.2 ng poly(A)⁺ RNA-derived), 1 μl of mmF (10 μM), 1 μl of mmR (10 μM), 5 μl of 10×reaction solution attached, 5 μl of dNTP (10 mM) and 0.5 μl of KlenTaq (TaKaRa), to which 36.5 μl of Otsuka's distilled water was added to make 50 μl in total. The reaction solution was subjected to PCR using Thermal Cycler 9600. PCR was conducted under the conditions of denaturation at 95° C. for 2 minutes followed by 38 repetitions of the cycle set to treat at 98° C. for 10 seconds, 65° C. for 10 seconds and 72° C. for 40 seconds. After it was confirmed by electrophoresis using an aliquot of the PCR product that the PCR product of about 1.1 kb was amplified, the PCR product was subcloned to E. coli, using TA cloning kit (Invitrogen). Using a plasmid extractor (Kurabo, Inc.), plasmids were extracted from the subcloned E. coli, and the base sequence of the inserted fragment was determined. It was thus conformed that the sequence was mouse type ML receptor cDNA. Next, the plasmid was digested with restriction enzymes SalI and NheI to give mouse type ML receptor cDNA fragment of about 1.1 kb. Furthermore, pAKKO-111H, which is an expression vector for animal cells, was digested with restriction eyzme sites SalI and NheI at the multicloning sites and then electrophoresed to recover the vector part. The mouse type ML receptor cDNA fragment and the expression vector prepared by the foregoing procedures were ligated, and E. coli JM109 was transformed to give E. coli JM109/pAKKOmML.

Transformant E. coli JM109/pAKKOmML was cultured to produce plasmid pAKKOmML DNA in large quantities.

After 20 μg out of the plasmid DNA was dissolved in 1 ml of saline (PBS), the solution was charged in a vial for gene transfer (Wako Junyaku K.K.) and vigorously agitated using a vortex mixer to form DNA-bearing liposomes.

CHO dhfr⁻ cells of 1 to 2×10⁶ were seeded on a Petri dish for cell culture of 35 mm in diameter and cultured for 20 hours. Then, the medium was replaced with fresh medium. The liposome solution of the amount (25 μl) corresponding to 0.5 μg of DNA was dropwise added to each dish, which was incubated for 16 hours to transfect the plasmid DNA.

The medium was further replaced with fresh medium followed by incubation for 1 day. The medium was further replaced with selective medium, and incubation was maintained for 3 days, which was digested with trypsin to disperse the cells. The cells were seeded on selective medium (minimum essential medium free of deoxyribonucleosides and ribonucleosides, or alpha medium supplemented with 10% dialyzed bovine serum) at a low density to select transformant. Only the transformant can grow on the selective medium; by repeating subculture, selection was repeated to establish mouse type ML receptor-expressing CHO cells or CHL-mML cells.

### Example 4

Detection of the Specific Activity of Releasing Arachidonic Acid Metabolites from CHO-mML Cells by Rat Cortistatin

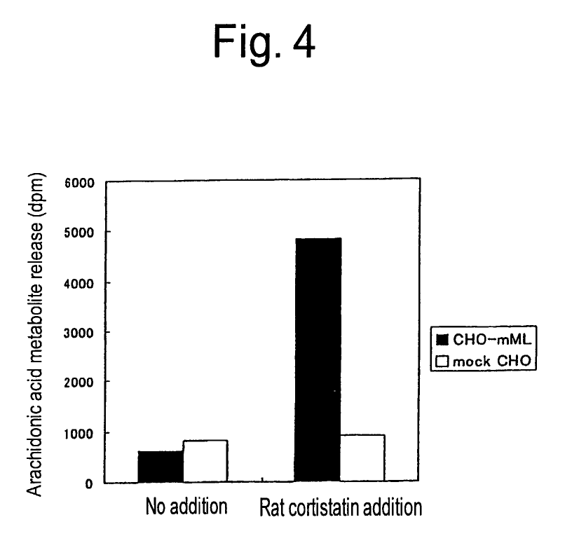

Mock CHO cells having introduced neither CHO-mML cells nor receptor gene were diluted in selective medium, and the diluted cells were seeded on a 24-well plate at a density of 0.5×10⁵ cells/0.5 ml/well, followed by incubation overnight at 37° C. under 5% CO₂. After the cells were washed twice with assay buffer (minimum essential medium free of deoxyribonucleosides and ribonucleosides, or alpha medium supplemented with 0.1% bovine serum albumin and 15 mM HEPES, pH 7.3), 0.5 ml of the assay buffer was added thereto, followed by preincubation for 30 minutes at 37° C. under 5% CO₂. The cells were again washed twice with 0.5 ml of the assay buffer, and 0.4 ml each of the assay buffer alone or a rat cortistatin (Peptide Research Institute, Catalog No. 4329-v) solution diluted with the assay buffer in a concentration of 1×10⁻⁴ M was added to the cells, followed by incubation for 30 minutes at 37° C. under 5% CO₂. After completion of the reaction, 0.3 ml of the reaction mixture was mixed with 3 ml of a liquid scintillator, and the radioactivity was measured with a scintillation counter to determine the amount of [³H] arachidonic acid metabolites released from the cells. As a result, an increase of the arachidonic acid metabolite release by rat cortistatin was noted only with the CHL-mML cells, showing that rat cortistatin functioned as an agonist to mouse type ML receptor and an increased reaction of arachidonic acid metabolites was observed in the signal transduction system (FIG. 4).

### INDUSTRIAL APPLICABILITY

The receptor protein of the present invention, its partial peptide or salts thereof as well as the polynucleotide encoding the same (e.g., DNA, RNA and derivatives thereof) can be used: (1) for determination of the ligand (agonist), (2) for acquisition of antibodies and antisera thereto, (3) for construction of the expression system of a recombinant receptor protein, (4) for screening development of the receptor-binding assay system using the expression system and screening of a candidate pharmaceutical compound, (5) for drug design based on comparison between ligands and receptors that are structurally analogous, (6) as a reagent for preparing a probe or a PCR primer in gene diagnosis, (7) for preparing a transgenic animal, or (8) as a pharmaceutical for the prevention/treatment in gene therapy, etc.

Based on the fact that the increased activity in the amount of releasing specific arachidonic acid metabolites from CHL-mML cells was noted by the addition of rat cortistatin, it is considered that when the receptor protein of the present invention such as mouse ML receptor, etc. is activated by the ligand binding, the phospholipase system would be activated in cells to increase a metabolism of phospholipids or an intracellular calcium ion level, thereby increasing the amount of the arachidonic acid metabolites released extracellularly. These results enable to determine a change in the amount of inositol phosphate production, a change in membrane phospholipids metabolism, a change in intracellular calcium ion level, the amount of arachidonic acid metabolite release, etc. using the cell capable of expressing the receptor protein of the present invention such as the ML receptor described above, thereby to explore endogenous ligands or screen agonists or antagonists.

1. A protein containing the same or substantially the same amino acid sequence as the amino acid sequence represented by SEQ ID NO: 1, or salts thereof.

2. The protein or salts thereof according to claim 1, wherein substantially the same amino acid sequence is represented by SEQ ID NO: 5.

3. A partial peptide of the protein according to claim 1, its esters or amides, or salts thereof.

4. A polynucleotide containing a polynucleotide encoding the protein according to claim 1 or the partial peptide according to claim 3.

5. The polynucleotide according to claim 4, which is a DNA.

6. The polynucleotide according to claim 4, which has the base sequence represented by SEQ ID NO: 2 or SEQ ID NO: 6.

7. A recombinant vector containing the polynucleotide according to claim 4.

8. A transformant transformed by the recombinant vector according to claim 7.

9. A method of manufacturing the protein or salts thereof according to claim 1 or the partial peptide, its amides or esters, or salts thereof according to claim 3, which comprises culturing the transformant according to claim 8 and producing the protein according to claim 1 or the partial peptide according to claim 3.

10. An antibody to the protein or salts thereof according to claim 1 or to the partial peptide, its esters or amides, or salts thereof according to claim 3.

11. The antibody according to claim 10, which is a neutralizing antibody to inactivate signal transduction of the protein according to claim 1.

12. A diagnostic product comprising the antibody according to claim 10.

13. A ligand to the protein or salts thereof according to claim 1 or to the partial peptide, its esters or amides, or salts thereof according to claim 3, which is obtainable using the protein or salts thereof according to claim 1 or using the partial peptide, its esters or amides, or salts thereof according to claim 3.

14. A pharmaceutical composition comprising the ligand according to claim 13.

15. A method of determining the ligand to the protein or salts thereof according to claim 1 or to the partial peptide, its esters or amides, or salts thereof according to claim 3, which comprises using the protein or salts thereof according to claim 1 or the partial peptide, its esters or amides, or salts thereof according to claim 3.

16. A method of screening a compound or salts thereof that alter the binding property between a ligand and the protein or salts thereof according to claim 1 or the partial peptide, its esters or amides, or salts thereof according to claim 3, which comprises using the protein or salts thereof according to claim 1, or the partial peptide, its esters or amides, or salts thereof according to claim 3.

17. A kit for screening a compound or salts thereof that alter the binding property between a ligand and the protein or salts thereof according to claim 1 or the partial peptide, its esters or amides, or salts thereof according to claim 3, comprising the protein or salts thereof according to claim 1, or the partial peptide, its esters or amides, or salts thereof according to claim 3.

18. A compound or salts thereof that alter the binding property between a ligand and the protein or salts thereof according to claim 1 or the partial peptide, its esters or amides, or salts thereof according to claim 3, which is obtainable using the screening method according to claim 16 or the screening kit according to claim 17.

19. A pharmaceutical composition comprising the compound or salts thereof according to claim 18.

20. A polynucleotide hybridizable to the polynucleotide according to claim 4 under high stringent conditions.

21. A polynucleotide comprising a base sequence complimentary to the polynucleotide according to claim 4, or a part of the base sequence.

22. A method of quantifying mRNA of the protein according to claim 1, which comprises using the polynucleotide according to claim 4, or a part of the polynucleotide.

23. A method of quantifying the protein according to claim 1 or the partial peptide, its amides or esters according to claim 3, or salts thereof, which comprises using the antibody according to claim 10.

24. A method for diagnosis of diseases associated with the functions of the protein according to claim 1, which comprises using the quantifying method according to claim 22 or 23.

25. A method of screening a compound or salts thereof that alter the expression level of the protein according to claim 1, which comprises using the quantifying method according to claim 22 or 23.

26. A method of screening a compound or salts thereof that alter the amount of the protein according to claim 1 on a cell membrane, which comprises using the quantifying method according to claim 23.


SEQUENCE LISTING (9 sequences)

SEQ ID NO: 1 - protein, 321 aa, Mouse
MEPLAMTLYPLESTQPTRNKTPNETTWSSEHTDDHTYFLVSLVICSLGLAGNGLLIWFLIFCIKRKPFTIYILHLAIADFMVLLCSSIMKLVNTFHIYNMTLESYAILFMIFGYNTGLHLLTAISVERCLSVLYPIWYQCQRPKHQSAVACMLLWALSVLVSGLENFFCILEVKPQFPECRYVYIFSCILTFLVFVPLMIFSNLILFIQVCCNLKPRQPTKLYVIIMTTVILFLVFAMPMKVLLIIGYYSSSLDDSVWDSLPYLNMLSTINCSINPIVYFVVGSLRRKRSRKSLKEALQKVFEEKPVVASRENVTQFSLPS

SEQ ID NO: 2 - DNA, 963 bp, Mouse
atggagccattggcaatgaccttgtatcctctggaatccacacaacccaccagaaacaaaaccccaaatgaaaccacctggtcttcagagcatacggatgaccacacttacttcttagtctccctggtcatttgttccctgggactggctgggaatggccttttgatatggttcctaatcttctgtatcaagaggaagccattcaccatctacatcctccatctcgccattgcagacttcatggtcctcctctgttcgtccatcatgaagctagtgaacacttttcacatctacaatatgaccttagagagctacgccatcctcttcatgatctttggctacaacacagggctccacctcctcacggccatcagcgtagagcggtgcctctccgtgctttatccaatctggtaccagtgccaacgcccaaaacaccagtccgctgtggcctgtatgctgctgtgggccctctctgttctcgtgtctggtttggaaaacttcttctgcattctggaagtgaagccccaattcccagaatgccgatacgtgtacatattctcctgtatcttgactttcctggtcttcgttcctctcatgatcttctctaatttgatcctcttcatccaagtctgctgtaacctgaagccacgtcaaccgaccaagctctatgtgatcatcatgaccacggtcatcctgtttcttgtcttcgccatgcccatgaaggtgttgcttatcatcggctactattctagttctcttgatgattctgtatgggactccctcccctacctgaacatgctgtccactataaactgcagcatcaacccaattgtctactttgtggtaggcagcctgaggaggaagaggagtaggaagtccctaaaagaagcactgcagaaagtcttcgaggaaaagccagtggtggcctcgagggagaatgtgacacagttctcgctgccttca

SEQ ID NO: 3 - DNA, 32 bp, Artificial Sequence
  primer
gtcgacgccacagagaaagccatcttcctgga

SEQ ID NO: 4 - DNA, 32 bp, Artificial Sequence
  primer
gctagcttccttggggatgtcctagctaaagg

SEQ ID NO: 5 - protein, 321 aa, Mouse
MEPLATTLCPQECTQTTRNETPNETTWSSEHVTKYTYISISLVICSLGLVGNGLLIWFLIFCIKRKPFTIYILHLAFADFMVLLCSSIIQLVNTFHIYDSTLVSYAVLFMIFGYNTGLHLLTAISVERCLSVLYPIWYHCRRPKHQSTVACTLLWALSVLVSGLENFFCILEVKPQFPECRYVYIFSCTLTFLVFVPLMVFSNLILFIQVCCNLKPRQPAKLYVIIMATVILFLVFAMPMKVLLIIGYYSNSTDASVWKSLPYLNMLSTINCSINPIVYFVVGSLRRKRSRKSLKEALQKVFEEKPVVASRENEVQFSLPL

SEQ ID NO: 6 - DNA, 963 bp, Mouse
atggagccattggcaacaaccttgtgtcctcaggaatgcacacaaaccaccagaaacgaaacccccaatgaaaccacctggtcttcagagcatgtgactaaatacacgtatatctccatctccctggtcatctgttcactgggactggttgggaacggccttttgatatggttcttgattttctgcatcaagaggaagccattcaccatctacatcctacatctcgcctttgctgacttcatggtcctcctctgttcatccatcattcagctagtgaacactttccacatttacgattccaccttagtgagctatgctgtcctcttcatgattttcggctacaacacgggcctgcacctcctcacggccatcagcgtggagcgctgcctctcggtgctttacccgatctggtaccactgccgacgcccgaaacaccagtccactgtggcctgtacgctgttgtgggccctctctgttcttgtgtctggtttggaaaacttcttctgcattctggaagtgaagccccagttcccagaatgccgatacgtgtacatattttcctgtaccttgactttcctggtcttcgtccctctcatggtcttctcgaacttgatactcttcatccaagtctgctgtaacctgaagccacgtcaaccagccaaactctatgtgatcatcatggccaccgtcatcctgttccttgtcttcgccatgcccatgaaggtgttgcttatcattggctactattccaattccactgatgcttctgtatggaaatctctcccctacctgaacatgctctccactataaactgcagcatcaacccaattgtctactttgtggtaggcagtctgaggaggaagaggagtaggaagtccctaaaagaagcactacagaaggtctttgaggaaaagccagtggtggcctcgagggagaatgaggtacaattctccctgccctta

SEQ ID NO: 7 - DNA, 30 bp, Artificial Sequence
  primer
atggagccattggcaacaaccttgtgtcct

SEQ ID NO: 8 - DNA, 30 bp, Artificial Sequence
  primer
tcataagggcagggagaattgtacctcatt

SEQ ID NO: 9 - protein, 14 aa, Rat
PCKNFFWKTFSSCK